(19) United States
(12) Patent Application Publication
(10) Pub. No.: US 2025/0035646 A1
(43) Pub. Date: Jan. 30, 2025

(54) BIOMARKERS FOR PROGNOSIS OF EARLY ONSET PREECLAMPSIA

(71) Applicants: B.R.A.H.M.S GMBH, Hennigsdorf (DE); UNIVERSITÉ LAVAL, Québec (CA)

(72) Inventor: Emmanuel BUJOLD, Québec (CA)

(73) Assignees: B.R.A.H.M.S GMBH, Hennigsdorf (DE); UNIVERSITÉ LAVAL, Québec (CA)

(21) Appl. No.: 18/717,027

(22) PCT Filed: Dec. 8, 2022
(86) PCT No.: PCT/EP2022/085007

§ 371 Date: Jun. 6, 2024

(30) Foreign Application Priority Data
Dec. 8, 2021 (EP) 21213234.4

(51) Int. Cl. G01N 33/68

(52) U.S. Cl. CPC G01N 33/689; G01N 2333/91205; G01N 2800/368; G01N 2800/52

(57) ABSTRACT

A method for treating a subject to reduce the risk of early onset preeclampsia in a pregnant subject determining a level of soluble fms-like tyrosine kinase-1 (sFlt-1) or fragment(s) thereof in a sample that has been isolated from said pregnant subject. The the sample is isolated from a subject before the end of the 12th week of gestation, where the level of sFlt-1 or fragment(s) thereof is indicative of early onset preeclampsia occurring before the end of the 33rd week of gestation. The subject is then treated to decrease the risk of developing, delay the time point of the onset and/or reduce the severity of preeclampsia.

The present invention is in the field of clinical and molecular diagnostics and prognostics for medical conditions, in particular for preeclampsia (PE).

The invention therefore relates to a method for the prognosis, prediction, risk assessment and/or risk stratification of preeclampsia in a pregnant subject, comprising determining a level of sFlt-1 or fragment(s) thereof in a sample that has been isolated from said pregnant subject; wherein said level of sFlt-1 or fragment(s) thereof is indicative of the likelihood of a preeclampsia.

The invention further relates to a method for the prognosis, prediction, risk assessment and/or risk stratification of early onset preeclampsia (EO-PE) in a pregnant subject, comprising determining a level of soluble fms-like tyrosine kinase-1 (sFlt-1) or fragment(s) thereof in a sample that has been isolated from said pregnant subject, wherein the sample is isolated from a subject before the end of the 12th week of gestation, wherein said level of sFlt-1 or fragment(s) thereof is indicative of the likelihood of early onset preeclampsia occurring before the end of the 33rd week of gestation.

The invention further relates to a method for the prognosis, prediction, risk assessment and/or risk stratification of early onset preeclampsia in a pregnant subject, comprising determining a level of soluble fms-like tyrosine kinase-1 (sFlt-1) or fragment(s) thereof and a level of placental growth factor (PlGF) or fragment(s) thereof in a sample that has been isolated from said pregnant subject. The invention further relates to the measurement of sFlt-1 and PlGF, combined with consideration of one or more additional factors selected from maternal age, body mass index, a uterine artery doppler measurement and/or mean arterial pressure (MAP).

The invention relates further to a kit for carrying out the method of the invention, comprising detection reagents for determining the level sFlt-1 or fragment(s) thereof, and optionally for determining the level of at least one additional biomarker as described herein such as PlGF, in a sample from a subject.

### BACKGROUND OF THE INVENTION

Preeclampsia (PE) is a pregnancy-specific hypertensive disorder and a leading cause of maternal and perinatal morbidity and death worldwide. The World Health Organization (WHO) estimates that 16% of global maternal mortality (˜63,000 maternal deaths annually) is due to PE alone. Infants are also at risk. Preeclampsia complicates approximately 2 to 8 percent of all pregnancies and is a major contributor to maternal and fetal mortality worldwide (Duley 2009, Semin Perinatal: 33: 130-37). Preeclampsia is generally defined as pregnancy associated or induced hypertension and proteinuria with onset after week 20 of gestation. Early onset preeclampsia (EO-PE) is a low prevalence subgroup of preeclampsia cases and occurs in 0.2-0.4% of all pregnancies, and is associated with various adverse perinatal outcomes, such as intra-uterine fetal death (IUFD) and high perinatal mortality.

The risk of maternal death is much higher in resource-limited settings. The most often recognized factor responsible for major maternal and fetal morbidity is failure to recognize preeclampsia in a timely manner. The result is that pregnant women cannot receive effective monitoring or treatment until long after complications associated with the disorder, including increased blood pressure and proteinuria, have developed. Additionally, pregnant women with little to no risk of developing such disorders must undergo unnecessary testing for symptoms throughout their pregnancy because there is no effective means by which caregivers may exclude them from risk in the early stages of pregnancy.

WO 2008/103202 A2 discloses a method of diagnosing a pregnancy related hypertensive disorder by means of measuring COMT, HIF-[alpha], EPO, LDH-A, ET-I, transferrin, transferrin receptor, and Flk-I, free VEGF, total VEGF, sFlt-1, PlGF. The altered expression of these polypeptides compared to the reference levels are an indicator of a pregnancy-related hypertensive disorder.

WO 2006/069373 A2 discloses a method of diagnosing a pregnant woman as having or being susceptible to developing a hypertensive disorder. The levels of sFlt-1 and placental growth factor (PlGF) in a urine sample are measured. The ratio of sFlt-1 expression to PlGF expression is used as an indicator as to whether the woman is at risk of developing a hypertensive disorder.

WO 2004/008946 A2 discloses a method of treating or preventing preeclampsia or eclampsia in a subject comprising the step of administering a compound capable of binding to soluble fms-like tyrosine kinase 1 (sFlt-1). It was further disclosed that the higher sFlt-1 concentrations in patients prior to onset of preeclampsia was due to acute rises in sFlt-1 within the 5 weeks before onset of clinical disease.

The most invasive treatment of preeclampsia until the present time is the termination of pregnancy either by premature vaginal or caesarean delivery. As mentioned above, maternal risks and fetal viability are significantly impaired in case of preeclampsia before gestational week 34. Accordingly, attempts should be made to delay delivery and to thereby improve survival of the newborn. Tsakiridis et al. (Volume 76, Number 10, Obstetrical and Gynecological Survey) highlighted the content of pregnancy related guidelines stating an early administration of low dose aspirin in high-risk patients, ideally in the first trimester until labor or 36 to 37 week of gestation.

Improving the prognosis within the 1^{st} trimester of preeclampsia in women who are clinically asymptomatic or suspected of having or developing preeclampsia is of significant clinical importance.

In the most severe forms, such as early-onset PE, it is necessary to initiate treatment as early as possible, ideally as early as the 11th week of pregnancy, although early treatment may also be initiated afterwards, such as in the 12^{th} to 15^{th} week of pregnancy. Currently, about 60-66% of preterm PE and about 70% of early-onset PE are detected with the FMF algorithm. The Fetal Medicine Foundation (FMF) screening algorithm includes consideration of multiple maternal characteristics and medical history, including factors such as blood pressure, pregnancy-associated plasma protein A and placenta growth factor, crown rump length, and uterine artery pulsatility index. The FMF algorithm is however complex and relies on uterine artery doppler measurements, a technique that is not commonly available to all pregnant subjects. Improved and simplified means for determining a risk of preeclampsia, in particular early onset preeclampsia, at an early stage of pregnancy are urgently required in the field.

### SUMMARY OF THE INVENTION

In light of the prior art the technical problem underlying the present invention is to provide improved or alternative means for the prognosis, prediction, risk assessment and/or risk stratification of preeclampsia in a pregnant subject. A further object of the invention is to provide means for the early prognosis or risk assessment of preeclampsia. A further object of the invention is to provide prognostic approaches towards risk assessment of preeclampsia within the first trimester of pregnancy. Further objects of the invention relate to providing means that improve and/or simplify screening or prognosis of early onset preeclampsia in early stages of pregnancy that increase sensitivity and preferably do not require uterine artery Doppler measurements.

This problem is solved by the features of the independent claims. Preferred embodiments of the present invention are provided by dependent claims.

The invention therefore relates to a method for the prognosis, prediction, risk assessment and/or risk stratification of early onset preeclampsia in a pregnant subject, comprising
a. determining a level of soluble fms-like tyrosine kinase-1 (sFlt-1) or fragment(s) thereof in a sample that has been isolated from said pregnant subject,
b. wherein the sample is isolated from a subject before the end of the 12th week of gestation (before 90 days GA),
c. wherein said level of sFlt-1 or fragment(s) thereof is indicative of the likelihood of early onset preeclampsia occurring before the end of the 33rd week of gestation.

The method described herein thus enables EO-PE risk assessment at an early stage of pregnancy, thus providing clinical practitioners the possibility of starting appropriate preventative treatment at a very early stage of pregnancy. In the most severe forms of PE, such as EO-PE, it is advised to initiate treatment as early as possible, preferably as early as the 11th week of pregnancy. Until the time of the invention molecular analyses were available for PE prognosis, although early-stage prognosis of EO-PE relied primarily on the FMF algorithm employing uterine artery Doppler measurements. The present invention thus enables a simple and reliable molecular diagnostic and/or prognostic approach towards identifying subjects at risk of EO-PE at a very early gestational age (GA).

Of note is that sFlt-1 measurement, when determined from multiple samples obtained throughout the entire first trimester, does not provide a statistically relevant correlation with EO-PE (FIG. 2, below). Of further note is that sFlt-1 measurement, when determined from multiple samples obtained after 12 6/7 weeks of GA, also does not provide a statistically relevant correlation with EO-PE (FIG. 3, FIG. 4, below). Surprisingly, the measurement of sFlt-1 within 12 weeks, or within 90 days of gestation, shows a significant correlation with EO-PE, in particular, an inverse correlation with EO-PE (FIG. 5, FIG. 6). Thus, determining sFlt-1 within 90 days GA enables reliable prognosis of EO-PE at an early time point.

The various aspects of the invention are unified by, benefit from, are based on and/or are linked by the common finding that the level of sFlt-1 or fragment(s) thereof, in samples from pregnant subjects of a GA before the end of 12 weeks, such as before 90 days GA, is indicative of the likelihood of early onset preeclampsia.

The present invention provides an efficient and reliable test for healthcare practitioners, such as doctors, nurses, personnel in emergency departments etc., to quickly and accurately assess the likelihood of a pregnant subject to develop PE. Typically, pregnant women with little to no risk of developing such disorders must undergo unnecessary testing for symptoms throughout their pregnancy because there is no effective means by which caregivers may exclude them from risk in the early stages of pregnancy.

High levels of placental soluble fms-like tryrosine kinase (sFlt-1) associate strongly with PE in the second-(14^{th} to 27^{th} week of gestation) and third-trimester (28^{th} week of gestation to birth) of pregnancy. Surprisingly, according to the present invention, the levels of sFlt-1 or fragments thereof in the first 90 days of pregnancy are also associated with early onset PE, whereby low sFlt-1 levels indicate EO-PE.

On the basis of this surprising finding, the present invention provides means for identifying pregnant subjects that have an increased or high risk of developing EO-PE and also identify patients that are less likely to develop such complications or in which the development of such complications can be practically ruled out, by determining the level of sFlt-1 or fragments thereof in a sample isolated from the patient.

A further advantage arising as an effect of this surprising finding is that besides the conventional combination of biomarkers, clinical parameters and imaging procedure for prognosing PE, the prognostic marker sFlt-1 can be employed in any medical setting regardless of whether a device for measuring uterine artery pulsatility index (UAPI) is available, for which a special sonographic device and an expert for operating the device and conducting the measurement is typically required. Therefore, a straightforward, minimally invasive test is enabled.

Of further note, the Aspirin for Evidence-Based Preeclampsia Prevention (ASPRE) trial, a multicenter trial including women identified at a high risk for preterm PE according to the FMF algorithm randomized to receive aspirin or placebo from 11 to 14 weeks of gestation to 37 weeks of gestation, showed a reduction of preterm PE with daily low-dose aspirin by 62% compared with the placebo group (relative risk, 0.38; 95% confidence interval [CI], 0.20-0.74).

This efficient treatment can now be applied to pregnant subjects at an early stage or pregnancy if they can be accurately prognosed with a high risk of developing EO-PE in the first 12 weeks of pregnancy. This leads to another advantage based on the surprising finding that a high risk of developing EO-PE can be prognosed in the first trimester of the pregnancy so that a precautionary mild treatment, medication or even bed rest and frequent monitoring instead of any serious measure e.g. early termination of the pregnancy after developing the PE in the later trimester of the pregnancy, can be taken for the subject prognosed with a high risk of developing PE.

In one embodiment, the method comprises additionally:
a. determining a level of placental growth factor (PlGF) or fragment(s) thereof in the sample that has been isolated from the subject,
b. wherein a combination of the level of sFlt-1 or fragment(s) thereof and said level of PlGF or fragment(s) thereof is indicative of the likelihood of early onset preeclampsia occurring before the end of the 33rd week of gestation.

As shown in more detail below, the combined use of sFlt-1 and PlGF leads to a statistically improved prognosis of EO-PE when samples are obtained early in pregnancy, for example before the end of the 12th week of gestation (before 90 days GA). Of note, is that PlGF is typically effective in prognosing EO-PE when measured after 90 days GA, whereas sFlt-1 appears to enable no reliable prognostic statements from a single measurement after 90 days GA (FIG. 7). Surprisingly, both sFlt-1 and PlGF enable an EO-PE prognosis when measured before the end of 12 weeks (within 90 days) GA, although sFlt-1 appears to provide greater sensitivity at comparable specificity values, preferably above 0.6 (FIG. 8). Also surprisingly, the combined analysis of sFlt-1 and PlGF shows an unexpected and synergistic enhancement in EO-PE prognosis when measured before 90 days GA (FIG. 9).

In embodiments of the method, the subject is in the 9th to 11th week of gestation.

In embodiments of the method, the subject is in the 11th week of gestation.

In embodiments of the method, the subject is at a gestational age (GA) of 50-90 days, preferably 60-90 days, or 70-90 days such as 63, 64, 65, 66, 67, 68, 69, 70, 71, 72, 73, 74, 75, 76, 77, 78, 79, 80, 81, 82, 83, 84, 85, 86, 87, 88, or 90 days GA.

As mentioned above, these early time points of sampling and testing enable early treatment initiation, which can be crucial in effectively addressing and/or preventing severe forms of PE occurring.

In embodiments, the method comprises additionally:
a. determining or providing maternal age, body mass index and/or a uterine artery doppler measurement of the subject,
b. wherein the combination of levels of sFlt-1 or fragment(s) thereof, preferably in combination with a level of PlGF or fragment(s) thereof, with maternal age, body mass index and/or a uterine artery doppler measurement of the subject, indicates early onset preeclampsia occurring before the end of the 33rd week of gestation.

In embodiments, the method comprises additionally:
a. determining or providing a level of mean arterial pressure (MAP) of the subject,
b. wherein the combination of levels of sFlt-1 or fragment(s) thereof, preferably in combination with a level of PlGF or fragment(s) thereof, with a level of MAP of the subject, indicates early onset preeclampsia occurring before the end of the 33rd week of gestation.

As shown in FIG. 10 below, the combination of sFlt-1 and PlGF measurements, when combined with an additional uterine artery Doppler measurement, shows a further improvement in diagnostic capabilities.

In embodiments, the method comprises:
a. determining a level of sFlt-1 or fragment(s) thereof, and determining a level of PlGF or fragment(s) thereof, in a sample that has been isolated from the subject, and
b. determining or providing maternal age, body mass index (BMI) and a uterine artery doppler measurement, and optionally mean arterial pressure (MAP), of the subject,
c. wherein a combination of said level of sFlt-1 or fragment(s) thereof, said level of PlGF or fragment(s) thereof, and maternal age, body mass index (BMI) and a uterine artery doppler measurement, and optionally mean arterial pressure (MAP), of the subject, is indicative of the likelihood of early onset preeclampsia occurring before the end of the 33rd week of gestation.

In embodiments, the method comprises:
a. determining a level of sFlt-1 or fragment(s) thereof, and determining a level of PlGF or fragment(s) thereof, in a sample that has been isolated from the subject, and
b. determining or providing maternal age, body mass index (BMI) and mean arterial pressure (MAP), and optionally a uterine artery doppler measurement of the subject,
c. wherein a combination of said level of sFlt-1 or fragment(s) thereof, said level of PlGF or fragment(s) thereof, and maternal age, body mass index (BMI) and mean arterial pressure (MAP), and optionally a uterine artery doppler measurement, of the subject, is indicative of the likelihood of early onset preeclampsia occurring before the end of the 33rd week of gestation.

In embodiments of the invention, the level of sFlt-1 or fragment(s) thereof determined in the sample is compared to a reference level, preferably a population average and/or median for a healthy population, wherein a level of sFlt-1 or fragment(s) thereof below or equal to the reference level is indicative of a risk, such as a high risk, of early onset preeclampsia.

In embodiments, the reference level is determined from a population of healthy pregnancies, or pregnancies that do not develop EO-PE, from the same GA.

In embodiments of the invention, the level of PlGF or fragment(s) thereof determined in the sample is compared to a reference level, preferably a population average and/or median for a healthy population, wherein a level of PlGF or fragment(s) thereof above the reference level is indicative of a risk, such as a high risk, of early onset preeclampsia.

In embodiments of the invention, the level of sFlt-1 or fragment(s) thereof, preferably wherein a combination of said level of sFlt-1 or fragment(s) thereof, said level of PlGF or fragment(s) thereof, and maternal age, body mass index (BMI) and a uterine artery doppler measurement, and optionally mean arterial pressure (MAP), of the subject, indicates an early onset of preeclampsia occurring from begin of 20th week of gestation and end of 33rd week of gestation.

Further embodiments and aspects of the invention relate to the method described herein used for indication of intra-uterine fetal death (IUFD).

Embodiments of the invention relate to the prognosis, prediction, risk assessment and/or risk stratification of IUFD. The prognosis of IUFD may be independent of, or in combination with, prognosing EO-PE. In other words, EO-PE may occur in combination with IUFD, or IUFD may occur independently of EO-PE. The features of the method and kits as described herein with respect to prognosis of EO-PE apply equally to the prognosis of IUFD, and vice versa.

As shown in more detail below, the combined use of sFlt-1 and PlGF leads to a prognosis of IUFD when the sample is obtained between 90-100 days GA (FIG. 11), and shows statistically improved prognosis of IUFD when the sample is obtained before 90 days GA (FIG. 12).

In embodiments of the invention, the level of sFlt-1 or fragment(s) thereof, preferably wherein a combination of said level of sFlt-1 or fragment(s) thereof, said level of PlGF or fragment(s) thereof, and maternal age, body mass index (BMI) and a uterine artery doppler measurement, and optionally mean arterial pressure (MAP), of the subject, indicates additionally the subsequent occurrence of intra-uterine fetal death (IUFD).

In embodiments of the invention, the sample is a bodily fluid sample, such as a blood sample, such as a venous blood sample, a capillary blood sample, a serum sample, a plasma sample, a vaginal fluid sample, a saliva sample or an amniotic fluid sample, preferably a blood, serum or plasma sample.

In embodiments of the invention, the level of sFlt-1 or fragment(s) thereof, and optionally a level of PlGF or fragment(s) thereof, maternal age, body mass index (BMI) a uterine artery doppler measurement, and/or mean arterial pressure (MAP) of the subject, indicates initiating or modifying a treatment of the subject to decrease the risk of developing, delay the time point of the onset and/or reduce the severity of preeclampsia, for example by balancing an angiogenetic/anti-angiogenetic process in placental development, lowering blood pressure and/or protect organ functions, such as of the kidney and/or liver.

In embodiments of the invention, the treatment is selected from the group consisting of one or more diuretics, beta-blockers, ace inhibitors, angiotensin II receptor blockers, calcium channel blockers, alpha-blockers, methyldopa, central agonists, and vasodilators, VEGF, PLGF, statins, arginine vasopressin receptor antagonist, L-arginine, citrulline, inhibitor of arginase (nor-NOHA), iron chelating agent (Deferoxamine), heparin, magnesium sulphate, diazepam, phenytoin, vitamin D, calcium, selenium inhibition of molecules, extracorporal extraction such as apharesis, life style recommendations, ambulant monitoring, increase the frequency of maternal and fetal monitoring, preferably low dose acetylsalicylic acid or metformin.

In embodiments of the invention, the treatment comprises administration of acetylsalicylic acid.

In embodiments of the invention, the treatment comprises administration of metformin.

The invention therefore relates to a method for treating a pregnant subject to reduce the risk of early onset preeclampsia, comprising
a. prognosis, prediction, risk assessment and/or risk stratification of early onset preeclampsia in a pregnant subject, comprising
   determining a level of soluble fms-like tyrosine kinase-1 (sFlt-1) or fragment(s) thereof in a sample that has been isolated from said pregnant subject,
   wherein the sample is isolated from a subject before the end of the 12th week of gestation (before 90 days GA),
   wherein said level of sFlt-1 or fragment(s) thereof is indicative of the likelihood of early onset preeclampsia occurring before the end of the 33rd week of gestation, and
b. administering to the subject a treatment to decrease the risk of developing, delay the time point of the onset and/or reduce the severity of preeclampsia.

In embodiments, the administered treatment relates to or comprises balancing an angiogenetic/anti-angiogenetic process in placental development, lowering blood pressure and/or protect organ functions, such as of the kidney and/or liver.

In embodiments of the method of treatment, the treatment is selected from the group consisting of one or more diuretics, beta-blockers, ace inhibitors, angiotensin II receptor blockers, calcium channel blockers, alpha-blockers, methyldopa, central agonists, and vasodilators, VEGF, PLGF, statins, arginine vasopressin receptor antagonist, L-arginine, citrulline, inhibitor of arginase (nor-NOHA), iron chelating agent (Deferoxamine), heparin, magnesium sulphate, diazepam, phenytoin, vitamin D, calcium, selenium inhibition of molecules, extracorporal extraction such as apharesis, life style recommendations, ambulant monitoring, increase the frequency of maternal and fetal monitoring, preferably low dose acetylsalicylic acid or metformin.

In embodiments of the method of treatment, the treatment comprises administration of acetylsalicylic acid.

In embodiments of the method of treatment, the treatment comprises administration of metformin.

In embodiments, the level of sFlt-1 or fragments thereof at least 6% lower than the reference sample indicates initiating or modifying a treatment of the subject to decrease the risk of developing, delay the time point of the onset or at least reduce the severity of PE such as balancing the angiogenetic/anti-angiogenetic process in the placental development, lowering blood pressure, protect organ functions such as from the kidney or liver.

In embodiments, the level of sFlt-1 or fragments thereof at least 8% lower than the reference sample, or 12% lower, 15% lower, or 20% lower than the reference sample, indicates initiating or modifying a treatment of the subject to decrease the risk of developing, delay the time point of the onset or at least reduce the severity of PE such as balancing the angiogenetic/anti-angiogenetic process in the placental development, lowering blood pressure, protect organ functions such as from the kidney or liver.

The gynecologist and/or physician are able to decide a suitable treatment for the subject according to the current conditions with or without risk factors. In embodiments, the level of sFlt-1 or fragments thereof at least 8% lower than the reference sample, or 12% lower, 15% lower, or 20% lower than the reference sample, indicates initiating or modifying a treatment of the subject to decrease the risk of developing, delay the time point of the onset or at least reduce the severity of PE such as balancing the angiogenetic/anti-angiogenetic process in the placental development, lowering blood pressure, protect organ functions such as from the kidney or liver.

The gynecologist and/or physician are able to decide a suitable treatment for the subject according to the current conditions with or without risk factors.

In embodiments, aspirin treatment can be initiated before 16 weeks of gestation which has been associated with a significant reduction of preterm PE. The Aspirin for Evidence-Based Preeclampsia Prevention (ASPRE) trial, a multicenter trial including women identified at a high risk for preterm PE according to the FMF algorithm randomized to receive aspirin or placebo from 11 to 14 weeks of gestation to 37 weeks of gestation, showed a reduction of preterm PE with daily low-dose aspirin by 62% compared with the placebo group (relative risk, 0.38; 95% confidence interval [CI], 0.20-0.74).

In embodiments of the invention, the subject has one or more risk factors, selected from the group consisting of hypothyroidism, hyperthyroidism, BMI over 24, first pregnancy, history of preeclampsia, ethnic with impaired risk, multiple pregnancy, migraines, lupus, blood coagulation disorder such as increased clotting, inflammatory diseases, cardiac preliminary disorders, diabetes, chronic kidney disease, and chronic hypertension.

In embodiments of the invention, the method additionally comprises determining a level of at least one additional biomarker or fragment(s) thereof in a sample from said patient, wherein the at least one additional biomarker is selected from the group consisting of beta hCG, Copeptin, Vasopressin, Troponin, BNP, ANP, CRP, trombocytes/leucocytes, IL6, IL11, MR-proADM, VEGF, PAPP-A, PlGF, Endoglin, pro-Epil, PP-13, ADAM-12, Vitamin D, Inhibin-a, Activin-a, Pentraxin-3, p-Selectin, free fetal Hemoglobin, alpha-1-Microglobulin, unconjugated Estriol, alpha-Fetoprotein, GDF15, Neurophysin2, LNPEP, ESM1, HGF, pikachurin, hemopexin, pp13, uE3, CT-proET1, ADAM12, sTNFαR1, RBP4, ICAM, cell free fetal DNA, FSTL3, Visfatin, AFP, MMP9, TIMP1, Flt1, PCT, SHGB, Creatinine, GBP1, IGFALS, urine protein, PAI1/PAI2, catechol-o-methyltransferase (COMT), breakdown products of heme (bilirubin, biliverdin, carbon monoxide, ferritin), breakdown products of arginine (urea, ornithine, citrulline, apolipoprotein H, arginosuccinic acid, ammonia), Uterine artery Doppler (UtA-Pi), diastolic Notch, MAP, blood pressure, smoking, leptin, genetic information and Arginine, wherein the level of the at least one additional biomarker and the level of sFlt-1 or fragment(s) thereof is indicative of early onset preeclampsia occurring before the end of the 33rd week of gestation.

In embodiments, additional marker(s) PAPP-A and/or PlGF are used in combination with sFlt-1.

In embodiments, additional marker(s) MAP, PAPP-A and/or PlGF are used in combination with sFlt-1.

In embodiments, additional marker(s) MAP, PAPP-A, beta hGC and/or PlGF are used in combination with sFlt-1.

In embodiments of the invention, the subject is nulliparous.

In embodiments of the invention, the subject had one or more former pregnancies.

In embodiments of the invention, the subject has a multiple pregnancy.

In embodiments of the invention, the subject is suspected of carrying a fetus with a chromosomal abnormality.

A further aspect of the invention relates to a kit for carrying out the method as described herein.

In embodiments, the kit comprises:
detection reagents for determining a level of sFlt-1 or fragment(s) thereof and/or for determining a level of PlGF or fragment(s) thereof in a sample from a subject, and
a computer readable medium and/or computer software in the form of computer executable code, configured to conduct an analysis useful in determining EO-PE risk.

In embodiments, the computer readable medium and/or computer software in the form of computer executable code is configured to:
i. compare a determined level of sFlt-1 or fragment(s) thereof and a determined level of PlGF or fragment(s) thereof, to one or more reference levels, preferably corresponding to a population average and/or median for a healthy population, and
ii. compare maternal age, body mass index, MAP and/or a uterine artery doppler measurement of the subject, to one or more reference levels, preferably corresponding to a population average and/or median for a healthy population.

In embodiments of the invention, the software in the kit, or the software with which the kit is configured to connect with, enables the comparison of determined molecular markers, such as those described herein, and provides a prognostic statement with respect to EO-PE risk based on samples obtained within 90 days GA.

In embodiments of the invention, the kit comprises a physical disk or computer readable medium with said software, alternatively, the kit provides a link or other code, such as a QR code, suitable to induce and/or provide connection with a server over the internet, where the appropriate software is maintained and/or can be executed.

### Additional Aspects and Embodiments of the Invention

The invention relates to a method for the prognosis, prediction, risk assessment and/or risk stratification of preeclampsia in a pregnant subject, comprising
a. determining a level of sFlt-1 or fragment(s) thereof in a sample that has been isolated from said pregnant subject
b. wherein said level of sFlt-1 or fragment(s) thereof is indicative of the likelihood of a preeclampsia.

The invention relates further to a kit for carrying out the method of the invention, comprising detection reagents for determining the level sFlt-1 or fragment(s) thereof, and optionally for determining the level of at least one additional biomarker as described herein, in a sample from a subject, wherein the kit additionally comprises a reference level, such as one or more cut-off levels, corresponding to a reference level indicative of a high or low risk of preeclampsia.

The invention therefore further relates to a method for the prognosis, prediction, risk assessment and/or risk stratification of preeclampsia in a pregnant subject, comprising
a. determining a level of sFlt-1 or fragment(s) thereof in a sample that has been isolated from said pregnant subject
b. wherein said level of sFlt-1 or fragment(s) thereof is indicative of the likelihood of a preeclampsia,
c. wherein the sample is isolated from a subject no later than the end of the 12^{th} week of pregnancy.

In one embodiment, the subject is in the 9^{th} to 11^{th} week of pregnancy, preferably in the 11^{th} week of pregnancy.

Surprisingly, a significant difference of predictive value (AUC value) of sFlt-1 for early-onset PE between women who were recruited in the 11th week of gestation, 12th week of gestation and 13^{th} week of gestation was observed. The AUC value for predicting early-onset PE of sFlt-1 level in the sample of a subject in 11^{th} week of gestation amounts to 0.82. The AUC value for predicting early-onset PE of sFlt-1 level in the sample of a subject in 12^{th} week of gestation amounts to 0.62. The AUC value for predicting early-onset PE of sFlt-1 level in the sample of a subject in 13^{th} week of gestation amounts to 0.50. A similar trend was surprisingly observed for the prediction of mid-onset PE.

The advantage based on the surprising finding is that the likelihood of developing PE can be accurately prognosed by means of determining the level of sFlt-1 or fragments thereof in early pregnancy, especially in 11^{th} week of gestation.

In embodiments, the maternal age is below 18.

In embodiments, the maternal age is 18-34.

In embodiments, the maternal age is above 34.

Surprisingly, a difference of predictive value (AUC value) of sFlt-1 for maternal age 18-34 and above 34 was observed.

Pregnant subjects with maternal age ≥34 years may have greater odds for preterm delivery, hypertension, superimposed PE, severe PE, and decreased risk for chorioamnionitis.

Pregnant subjects with maternal age ≥40 years may have increased odds for mild PE, fetal distress, and poor fetal growth.

It is of great advantage that the subjects of the indicated risk groups, comprising the maternal age below 18, greater or equal to 34, and greater or equal to 40, may receive an accurate prognosis of preeclampsia at a time point before or until the end of the 12th week of gestation.

In embodiments, the method as described herein comprises:
a. the level of sFlt-1 or fragment(s) thereof determined in the sample is compared to a reference level which is derived from a reference sample.
b. wherein a level of sFlt-1 or fragment(s) thereof below or equal to the reference level is indicative of high risk of preeclampsia, or
c. a level of sFlt-1 or fragment(s) thereof above a reference level is indicative of low risk of preeclampsia.

In embodiments, the reference level is derived from a reference sample which is isolated from a pregnant subject not having or getting any pregnancy related hypertensive disorder, such as PE or eclampsia.

In embodiments, the level of sFlt-1 or fragments thereof at least 2-5% lower than the reference sample is indicative of a high risk of PE. In embodiments, the level of sFlt-1 or fragments thereof less than 2% lower than the reference sample is indicative of a low risk of PE.

In embodiments, the level of sFlt-1 or fragments thereof at least 5.8% lower than the reference sample is indicative of a high risk of PE. In embodiments, the level of sFlt-1 or fragments thereof less than 5.8% lower than the reference sample is indicative of a low risk of PE.

In embodiments, the level of sFlt-1 or fragments thereof at least 8% lower than the reference sample is indicative of a high risk of PE. In embodiments, the level of sFlt-1 or fragments thereof less than 8% lower than the reference sample is indicative of a low risk of PE.

In embodiments, the level of sFlt-1 or fragments thereof at least 12% lower than the reference sample is indicative of a high risk of PE. In embodiments, the level of sFlt-1 or fragments thereof less than 12% lower than the reference sample is indicative of a low risk of PE.

In embodiments, the level of sFlt-1 or fragments thereof at least 15% lower than the reference sample is indicative of a high risk of PE. In embodiments, the level of sFlt-1 or fragments thereof less than 15% lower than the reference sample is indicative of a low risk of PE.

In embodiments, the level of sFlt-1 or fragments thereof at least 20% lower than the reference sample is indicative of a high risk of PE. In embodiments, the level of sFlt-1 or fragments thereof less than 20% lower than the reference sample is indicative of a low risk of PE.

In embodiments, the level of sFlt-1 or fragments thereof at least 5.8% lower than the reference sample is indicative of a high risk of term PE. In embodiments, the level of sFlt-1 or fragments thereof less than 5.8% lower than the reference sample is indicative of a low risk of term PE.

In embodiments, the level of sFlt-1 or fragments thereof at least 8.8% lower than the reference sample is indicative of a high risk of term PE. In embodiments, the level of sFlt-1 or fragments thereof less than 8.8% lower than the reference sample is indicative of a low risk of term PE.

In embodiments, the level of sFlt-1 or fragments thereof at least 11% lower than the reference sample is indicative of a high risk of term PE. In embodiments, the level of sFlt-1 or fragments thereof less than 11% lower than the reference sample is indicative of a low risk of term PE.

In embodiments, the level of sFlt-1 or fragments thereof at least 14.7% lower than the reference sample is indicative of a high risk of preterm PE. In embodiments, the level of sFlt-1 or fragments thereof less than 14.7% lower than the reference sample is indicative of a low risk of preterm PE.

In embodiments, the level of sFlt-1 or fragments thereof at least 16.7% lower than the reference sample is indicative of a high risk of preterm PE. In embodiments, the level of sFlt-1 or fragments thereof less than 16.7% lower than the reference sample is indicative of a low risk of preterm PE.

In embodiments, the level of sFlt-1 or fragments thereof at least 20% lower than the reference sample is indicative of a high risk of preterm PE. In embodiments, the level of sFlt-1 or fragments thereof less than 20% lower than the reference sample is indicative of a low risk of preterm PE.

In embodiments, the level of sFlt-1 or fragment(s) thereof indicates an early onset of PE occurring from the beginning of the 20^{th} week of gestation to the end of the 33rd week of gestation, or a mid-onset of PE occurring from the beginning of the 34^{th} week of gestation to the end of the 36^{th} week of gestation.

In embodiments, the level of sFlt-1 or fragments thereof in a sample at least 15%, preferably 20% lower than the reference level is indicative of early onset of PE occurring from the beginning of the 20^{th} week of gestation to the end of the 33rd week of gestation.

In embodiments, the level of sFlt-1 or fragments thereof in a sample less than 15% lower than the reference level is indicative of low risk of early onset of PE.

In embodiments, the level of sFlt-1 or fragments thereof in a sample at least 6%, preferably 12% lower than the reference level is indicative of early onset of PE occurring from the beginning of the 20^{th} week of gestation to the end of the 33rd week of gestation, or mid-onset of PE occurring from the beginning of the 34^{th} week of gestation to the end of the 36^{th} week of gestation.

In embodiments, a further risk parameter is the gender of the fetus. Surprisingly, a significant difference of the predictive value (AUC value) of an sFlt-1 level in a sample of the subject with at least a female fetus or at least a male fetus, as well as a significant reduction of sFlt-1 level compared to the reference level, can be observed. In embodiments, the level of sFlt-1 in the sample of the subject is reduced at least 6%, 7%, 8%, 9%, 10%, 11%, 12%, 13%, 14%, 15%, 16%, 17%, 18%, 19%, 20%, 21%, 22%, 23%, 24% or 25% compared to the reference level. Based on this surprising finding, the subject carrying such a risk parameter can be prognosed with a risk of PE.

One aspect of the invention relates to a kit for carrying out the method of the invention, comprising
detection reagents for determining the level sFlt-1 or fragment(s) thereof, and optionally for determining the level of at least one additional biomarker as described herein, preferably PAPP-A and/or PlGF, in a sample from a subject, and
a reference level, such as one or more cut-off levels, corresponding to a reference level indicative of a high or low risk of preeclampsia.

In one embodiment, a kit for carrying out the method as described herein, comprises
detection reagents for determining the level sFlt-1 or fragment(s) thereof, and optionally for determining the level of at least one additional biomarker as described herein, preferably PAPP-A and/or PlGF, in a sample from a subject, and
a reference level, such as one or more cut-off levels, corresponding to
   a level of sFlt-1 or fragment(s) thereof in a sample isolated from a subject up to 18 years old indicative of a risk of preeclampsia,
   a level of sFlt-1 or fragment(s) thereof in a sample isolated from a subject from 18-34 years old indicative of a risk of preeclampsia, or
   a level of sFlt-1 or fragment(s) thereof in a sample isolated from a subject older than 34 years old indicative of a risk of preeclampsia,
wherein said reference level is stored on a computer readable medium and/or employed in the form of computer executable code configured for comparing the determined levels of sFlt-1 or fragment(s) thereof, and optionally the determined levels of at least one additional biomarker, to said reference level.

In one embodiment, a kit for carrying out the method as described herein, comprises
detection reagents for determining the level sFlt-1 or fragment(s) thereof, and optionally for determining the level of at least one additional biomarker as described herein, preferably PAPP-A and/or PlGF, in a sample from a subject, and
a reference level, such as one or more cut-off levels, corresponding to
   a level of sFlt-1 or fragment(s) thereof in a sample isolated from a subject with blood group AB indicative of a risk of preeclampsia, or
   a level of sFlt-1 or fragment(s) thereof in a sample isolated from a subject with blood group Rh negative indicative of a risk of preeclampsia, or
   a level of sFlt-1 or fragment(s) thereof in a sample isolated from a subject with blood group Rh negative and with a biological father of the fetus with Rh positive indicative of a risk of preeclampsia, or
   a level of sFlt-1 or fragment(s) thereof in a sample isolated from a subject carrying at least one female fetus indicative of a risk of preeclampsia, or
   a level of sFlt-1 or fragment(s) thereof in a sample isolated from a subject carrying at least one male fetus indicative of a risk of preeclampsia, or
   a level of sFlt-1 or fragment(s) thereof in a sample isolated from a subject who is nulliparous indicative of a risk of preeclampsia, or
   a level of sFlt-1 or fragment(s) thereof in a sample isolated from a subject having one or more former pregnancies indicative of a risk of preeclampsia, or
   a level of sFlt-1 or fragment(s) thereof in a sample isolated from a subject with multiple pregnancy indicative of a risk of preeclampsia, or
   a level of sFlt-1 or fragment(s) thereof in a sample isolated from a subject suspected of carrying a fetus with a chromosomal abnormality indicative of a risk of preeclampsia,
wherein said reference level is stored on a computer readable medium and/or employed in the form of computer executable code configured for comparing the determined levels of sFlt-1 or fragment(s) thereof, and optionally the determined levels of at least one additional biomarker, to said reference level.

In one embodiment, a kit for carrying out the method as described herein, comprises
detection reagents for determining the level sFlt-1 or fragment(s) thereof, and optionally for determining the level of at least one additional biomarker as described herein, preferably PAPP-A and/or PlGF, in a sample from a subject, and
a reference level, such as one or more cut-off levels, corresponding to
   a level of sFlt-1 or fragment(s) thereof in a sample isolated from a subject indicative of a risk of early onset of preeclampsia occurring from the beginning of the 20th week of gestation to the end of the 33rd week of gestation,
   a level of sFlt-1 or fragment(s) thereof in a sample isolated from subject indicative of a risk of preeclampsia of mid-onset of preeclampsia occurring from the beginning of the 34th week of gestation to the end of the 36th week of gestation, or
   a level of sFlt-1 or fragment(s) thereof in a sample isolated from subject indicative of a low risk of developing preeclampsia,
wherein said reference level is stored on a computer readable medium and/or employed in the form of computer executable code configured for comparing the determined levels of sFlt-1 or fragment(s) thereof, and optionally the determined levels of at least one additional biomarker, to said reference level.

The invention relates further to a method for identifying subjects at risk of early onset preeclampsia (occurring before the end of the 33rd week of gestation) and treating said subjects, the method comprising:
(a) diagnosis, prognosis, prediction, risk assessment and/or risk stratification of early onset preeclampsia in a pregnant subject, comprising:
   determining a level of soluble fms-like tyrosine kinase-1 (sFlt-1) or fragment(s) thereof in a sample that has been isolated from said pregnant subject,
   wherein the sample is isolated from a subject before the end of the 12th week of gestation (before 90 days GA),
   wherein said level of sFlt-1 or fragment(s) thereof is indicative of the likelihood of early onset preeclampsia occurring before the end of the 33rd week of gestation, and
(b) administering to said subject a treatment for or to reduce the risk of early onset preeclampsia.

The invention relates further to a method for detecting soluble fms-like tyrosine kinase-1 (sFlt-1) or fragment(s) thereof in a sample from a subject, the method comprising:
providing a sample of a subject, preferably a blood sample or sample derived from a blood sample, having a complex comprising at least one binder to sFlt-1 or fragment(s) thereof; and preferably providing a sample of a subject, preferably a blood sample or sample derived from a blood sample, having a complex comprising at least one binder to PlGF or fragment(s) thereof;
wherein the sample has a level of sFlt-1, and preferably of PlGF, that is below or above a threshold value, such as any threshold disclosed herein, preferably a population mean and/or a population median of sFlt-1 levels from a normal pregnancy at any given time point in the respective patient population.

The invention relates further to a method for treating and/or reducing the risk of early onset preeclampsia occurring before the end of the 33rd week of gestation, or for administering to a subject a treatment for early onset preeclampsia, the method comprising:
administering to a subject a treatment for early onset preeclampsia,
wherein said subject has been determined to have, in a bodily fluid sample of the subject, preferably a blood sample or sample derived from a blood sample, a level of sFlt-1, and preferably of PlGF, that is below or above a threshold value, such as any threshold disclosed herein, preferably a population mean and/or a population median of sFlt-1, and preferably of PlGF, levels from a normal pregnancy at any given time point in the respective patient population.

The embodiments describing a method of the invention may be used to describe the kit of the invention, and vice versa. Features disclosed in some embodiments of the method may also be used to characterize other embodiments of the method or other methods of the invention. The invention is unified by the novel and beneficial employment of the prognostic marker sFlt-1 for indicating the risk of developing EO-PE in samples obtained until 12 weeks (90 days) GA, and thus the relevant features described herein for one aspect may be used to describe any given aspect of the invention, in a manner in conformity with the understanding of a skilled person.

### DETAILED DESCRIPTION OF THE INVENTION

All cited documents of the patent and non-patent literature are hereby incorporated by reference in their entirety.

The present invention relates to a method for the prognosis, prediction, risk assessment and/or risk stratification of preeclampsia in a pregnant subject, comprising a) determining a level of sFlt-1 or fragment(s) thereof in a sample that has been isolated from said pregnant subject, b) wherein said level of sFlt-1 or fragment(s) thereof is indicative of the likelihood of a preeclampsia.

As used herein, the term “subject” shall mean a mammal, including, but not limited to, a human or non-human mammal, such as a bovine, equine, canine, ovine, or feline. Included in this definition are pregnant, post-partum, and non-pregnant mammals.

In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification.

As used herein, “prognosis” relates to the prediction of an outcome or a specific risk for a subject developing PE. This may also include an estimation of the chance of recovery or the chance of an adverse outcome for said subject. Also the assessment of the severity of the PE may be encompassed by the term “prognosis” or “risk assessment” or “risk stratification”.

As used herein, “preeclampsia” (PE) is used in its ordinary meaning. PE can be defined according to well established criteria, such as a blood pressure of at least 140/90 mm Hg and urinary excretion of at least 0.3 grams of protein in a 24-hour urinary protein excretion (or at least +1 or greater on dipstick testing), each on two occasions 4-6 hours apart.

Preeclampsia is considered a multi-system disorder that is characterized by hypertension with proteinuria or edema, or both, glomerular dysfunction, brain edema, liver edema, or coagulation abnormalities due to pregnancy or the influence of a recent pregnancy. Preeclampsia generally occurs after the 20th week of gestation. Preeclampsia is generally defined as some combination of the following symptoms: (1) a systolic blood pressure (BP) >140 mmHg and a diastolic BP >90 mmHg after 20 weeks gestation (generally measured on two occasions, 4-168 hours apart), (2) new onset proteinuria (1+ by dipstick on urinalysis, >300 mg of protein in a 24-hour urine collection, or a single random urine sample having a protein/creatinine ratio >0.3), and (3) resolution of hypertension and proteinuria by 12 weeks postpartum. Severe pre-eclampsia is generally defined as (1) a diastolic BP >110 mmHg (generally measured on two occasions, 4-168 hours apart) or (2) proteinuria characterized by a measurement of 3.5 g or more protein in a 24-hour urine collection or two random urine specimens with at least 3+ protein by dipstick.

In preeclampsia, hypertension and proteinuria generally occur within seven days of each other. In severe preeclampsia, severe hypertension, severe proteinuria and HELLP syndrome (hemolysis, elevated liver enzymes, low platelets) or eclampsia can occur simultaneously or only one symptom at a time. Occasionally, severe pre-eclampsia can lead to the development of seizures. This severe form of the syndrome is referred to as eclampsia. “Eclampsia” can also include dysfunction or damage to several organs or tissues such as the liver (e.g., hepatocellular damage, periportal necrosis) and the central nervous system (e.g., cerebral edema and cerebral hemorrhage). The etiology of the seizures is thought to be secondary to the development of cerebral edema and focal spasm of small blood vessels in the kidney.

“Severe preeclampsia” or “high severity of preeclampsia” is also defined in accordance with established criteria, as a blood pressure of at least 160/110 mm Hg on at least 2 occasions 6 hours apart and greater than 5 grams of protein in a 24-hour urinary protein excretion or persistent +3 proteinuria on dipstick testing.

Severe preeclampsia may include HELLP syndrome (hemolysis, elevated liver enzymes, low platelet count). Other elements of severe preeclampsia may include in-utero growth restriction (IUGR) in less than the 10% percentile according to the US demographics, persistent neurologic symptoms (headache, visual disturbances), epigastric pain, oliguria (less than 500 mL/24 h), serum creatinine greater than 1.0 mg/dL, elevated liver enzymes (greater than two times normal), thrombocytopenia (<100,000 cells/[mu]L).

As used herein “preterm birth” is defined as delivery before 37 weeks of gestation.

As used herein “preterm PE” is defined as PE with delivery before 37 weeks of gestation.

As used herein, “early onset of preeclampsia” shall mean the symptoms of preeclampsia occur from the beginning of the 20^{th} week of gestation to the end of the 33^{rd} week of gestation. In embodiments, early onset of preeclampsia refers to cases with delivery before 34 weeks of gestation.

As used herein, “mid-onset of preeclampsia” shall mean the symptoms of preeclampsia occur from the beginning of the 34^{th} week of gestation to the end of the 36^{th} week of gestation.

As used herein, “late onset of preeclampsia” shall mean the symptoms of preeclampsia occur from the 37^{th} week of gestation.

By way of example, “symptoms of preeclampsia” may refer to the following: (1) a systolic blood pressure (BP) >140 mmHg and a diastolic BP >90 mmHg after 20 weeks gestation, (2) new onset proteinuria (1+ by dipstick on urinalysis, >300 mg of protein in a 24 hour urine collection, or random urine protein/creatinine ratio >0.3), and (3) resolution of hypertension and proteinuria by 12 weeks postpartum. The symptoms of preeclampsia can also include renal dysfunction and glomerular endotheliosis or hypertrophy.

As used herein, “symptoms of eclampsia” refers to the development of any of the following symptoms due to pregnancy or the influence of a recent pregnancy: seizures, coma, thrombocytopenia, liver edema, pulmonary edema, and cerebral edema.

“At risk of developing” a pregnancy-related hypertensive disorder such as preeclampsia or eclampsia refers to a subject who does not currently have, but has a greater than average chance of developing, a pregnancy-related hypertensive disorder. Such at risk subjects include pregnant subject with an sFlt-1 level in blood reduced, without limitation, at least by 6%, 7%, 8%, 9%, 10%, 11%, 12%, 13%, 14%, 15%, 16%, 17%, 18%, 19%, 20%, 21%, 22, 23%, 24% or 25% compared to the reference level. Patients may in some embodiments show no other signs of a pregnancy-related hypertensive disorder such as preeclampsia.

As used herein, the term “indicative of the likelihood of a preeclampsia” refers to a subject who does not currently have, but has a greater than average chance of developing a preeclampsia. Such prognosis or risk assessment is based on the determined level of sFlt-1 or fragment(s) thereof. The level of sFlt-1 or fragment(s) thereof is indicative of the chance of developing a preeclampsia. In one embodiment, the level of sFlt-1 or fragment(s) thereof determined in the sample is compared to a reference level, wherein a level of sFlt-1 or fragment(s) thereof below or equal to the reference level is indicative of high risk of preeclampsia or a level of sFlt-1 or fragment(s) thereof above a reference level is indicative of low risk of preeclampsia.

As used herein, “high risk of preeclampsia” shall mean high risk of developing preeclampsia, but does not currently have preeclampsia.

As used herein, “low risk of preeclampsia” shall mean low risk of developing preeclampsia, but does not currently have preeclampsia.

“Pregnancy-related hypertensive disorder” shall mean any condition or disease during pregnancy that is associated with or characterized by an increase in blood pressure. Included among these conditions and diseases are preeclampsia (including premature preeclampsia, severe preeclampsia), eclampsia, gestational hypertension, HELLP syndrome, (hemolysis, elevated liver enzymes, low platelets), abruption placenta, chronic hypertension during pregnancy, pregnancy with intra uterine growth restriction, and pregnancy with a small for gestational age (SGA) infant.

As used herein, the term “soluble Flt-1 (sFlt-1)” (soluble fms-like tyrosine kinase 1, also known as sVEGF-R1) refers to the soluble form of the Flt-1 receptor, that is homologous to the protein defined by GenBank accession number U01134 or UniProt P17948 or entry name VGFR1_HUMAN and that has sFlt-1 biological activity. The biological activity of an sFlt-1 polypeptide may be assayed using any standard method, for example, by assaying sFlt-1 binding to VEGF. sFlt-1 lacks the transmembrane domain and the cytoplasmic tyrosine kinase domain of the Flt-1 receptor. sFlt-1 can bind to VEGF and PlGF bind with high affinity, but it cannot induce proliferation or angiogenesis and is therefore functionally different from the Flt-1 and KDR receptors. sFlt-1 was initially purified from human umbilical endothelial cells and later shown to be produced by trophoblast cells in vivo. As used herein, sFlt-1 includes any sFlt-1 family member or isoform.

As used herein, the term “specifically binds” refers to a compound or antibody or any detection reagent which recognizes and binds a polypeptide, i.e. sFlt-1 or any fragment(s) thereof but that does not substantially recognize and bind other molecules in a sample, for example, a biological sample, which naturally includes a polypeptide of sFLt-1 or any fragment(s) thereof. In one embodiment, an antibody that specifically binds sFlt-1 does not bind Flt-1.

As used herein, the “detection reagent” or the like are reagents that are suitable to determine the herein described marker(s), e.g. of sFlt-1, PAPP-A, PlGF. Such exemplary detection reagents are, for example, ligands, e.g. antibodies or fragments thereof, which specifically bind to the peptide or epitopes of the herein described marker(s). Such ligands might be used in immunoassays as described above. Further reagents that are employed in the immunoassays to determine the level of the marker(s) may also be comprised in the kit and are herein considered as detection reagents. Detection reagents can also relate to reagents that are employed to detect the markers or fragments thereof by mass spectrometry-based methods. Such detection reagent can thus also be reagents, e.g. enzymes, chemicals, buffers, etc, that are used to prepare the sample for the MS analysis. A mass spectrometer can also be considered as a detection reagent. Detection reagents according to the invention can also be calibration solution(s), e.g. which can be employed to determine and compare the level of the marker(s).

According to the invention, the antibodies may be monoclonal as well as polyclonal antibodies. Particularly, antibodies that are specifically binding to at least sFlt-1 or fragments thereof are used.

An antibody is considered to be specific, if its affinity towards the molecule of interest, e.g. sFlt-1, or the fragment thereof is at least 50-fold higher, preferably 100-fold higher, most preferably at least 1000-fold higher than towards other molecules comprised in a sample containing the molecule of interest. It is well known in the art how to develop and to select antibodies with a given specificity. In the context of the invention, monoclonal antibodies as detection reagent are preferred. The antibody or the antibody binding fragment binds specifically to the herein defined markers or fragments thereof. In particular, the antibody or the antibody binding fragment binds to the herein defined peptides of sFlt-1. Thus, the herein defined peptides can also be epitopes to which the antibodies specifically bind. Further, an antibody or an antibody binding fragment is used in the methods and kits of the invention that binds specifically to sFlt-1 or fragments thereof.

Further, an antibody or an antibody binding fragment is used in the methods and kits of the invention that binds specifically to sFlt-1 or fragments thereof and optionally to other markers of the present inventions such as PAPP-A or PlGF.

Exemplary immunoassays can be luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads-based arrays, protein microarray assays, rapid test formats, rare cryptate assay. Further, assays suitable for point-of-care testing and rapid test formats such as for instance immune-chromatographic strip tests can be employed. Automated immunoassays are also intended, such as the B⋅R⋅A⋅H⋅M⋅S KRYPTOR assay.

Alternatively, instead of antibodies, other capture molecules or molecular scaffolds that specifically and/or selectively recognize sFlt-1 may be encompassed by the scope of the present invention. Herein, the term “capture molecules” or “molecular scaffolds” comprises molecules which may be used to bind target molecules or molecules of interest, i.e. analytes (e.g. sFlt-1), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecules or molecules of interest. In the context of the present invention, capture molecules or molecular scaffolds may for instance be selected from the group consisting of a nucleic acid molecule, a carbohydrate molecule, a PNA molecule, a protein, a peptide and a glycoprotein. Capture molecules or molecular scaffolds include, for example, aptamers, DARpins (Designed Ankyrin Repeat Proteins). Affimers and the like are included.

The method according to the present invention can furthermore be embodied as a homogeneous method, wherein the sandwich complexes formed by the antibody/antibodies and the marker, sFlt-1 or a fragment thereof, which is to be detected remains suspended in the liquid phase. In this case it is preferred, that when two antibodies are used, both antibodies are labeled with parts of a detection system, which leads to generation of a signal or triggering of a signal if both antibodies are integrated into a single sandwich. Such techniques are to be embodied in particular as fluorescence enhancing or fluorescence quenching detection methods. A particularly preferred aspect relates to the use of detection reagents which are to be used pair-wise, such as for example the ones which are described in U.S. Pat. No. 4,882,733, EP0180492 or EP0539477 and the prior art cited therein. In this way, measurements in which only reaction products comprising both labeling components in a single immune-complex directly in the reaction mixture are detected, become possible. For example, such technologies are offered under the brand names TRACE™ (Time Resolved Amplified Cryptate Emission) or KRYPTOR™, implementing the teachings of the above-cited applications. Therefore, in particular preferred aspects, a diagnostic device is used to carry out the herein provided method. For example, the level of sFlt-1 or fragments thereof and/or the level of any further marker of the herein provided method, such as PAPP-A, PlGF, is determined. In particular preferred aspects, the diagnostic device is the B⋅R⊇A⋅H⋅M⋅S KRYPTOR.

In embodiments, a quantitative determination of sFlt-1 can be performed by automated immunofluorescent assay B⋅R⊇A⋅H⋅M⋅S sFlt-1 KRYPTOR assay preferably together with the B⋅R⋅A⋅H⋅M⋅S PlGF plus KRYPTOR assay. With the lower and upper detection limits of 22 and 90 000 μg/mL B⋅R⋅A⋅H⋅M⋅S sFlt-1 KRYPTOR provides the measuring range needed for a reliable detection of clinical sFlt-1 values throughout pregnancy. Only 8 μL serum sample isolated from the subject are needed for the assay.

A skilled person is capable of obtaining or developing means for the identification, measurement, determination and/or quantification of any one of the above sFlt-1 molecules, or fragments or variants thereof, as well as the other markers of the present invention according to standard molecular biological practice.

The level of the marker of the present invention, e.g. the sFlt-1 or fragments thereof, PAPP-A or fragments thereof, or other markers, can also be determined by a mass spectrometric (MS) based methods. Such a method may comprise detecting the presence, amount or concentration of one or more modified or unmodified fragment peptides of e.g. sFlt-1 or the PAPP-A, PlGF in said biological sample or a protein digest (e.g. tryptic digest) from said sample, and optionally separating the sample with chromatographic methods, and subjecting the prepared and optionally separated sample to MS analysis. For example, selected reaction monitoring (SRM), multiple reaction monitoring (MRM) or parallel reaction monitoring (PRM) mass spectrometry may be used in the MS analysis, particularly to determine the amounts of sFlt-1 or fragments thereof.

Herein, the term “mass spectrometry” or “MS” refers to an analytical technique to identify compounds by their mass. In order to enhance the mass resolving and mass determining capabilities of mass spectrometry, the samples can be processed prior to MS analysis.

Accordingly, the invention relates to MS detection methods that can be combined with immuno-enrichment technologies, methods related to sample preparation and/or chromatographic methods, preferably with liquid chromatography (LC), more preferably with high performance liquid chromatography (HPLC) or ultra-high performance liquid chromatography (UHPLC).

Sample preparation methods comprise techniques for lysis, fractionation, digestion of the sample into peptides, depletion, enrichment, dialysis, desalting, alkylation and/or peptide reduction. However, these steps are optional. The selective detection of analyte ions may be conducted with tandem mass spectrometry (MS/MS). Tandem mass spectrometry is characterized by mass selection step (as used herein, the term “mass selection” denotes isolation of ions having a specified m/z or narrow range of m/z/s), followed by fragmentation of the selected ions and mass analysis of the resultant product (fragment) ions.

As used herein, the term “detection reagent specifically binding sFlt-1 and fragment(s) thereof” shall mean the detection reagent recognizes and binds a polypeptide of sFLT-1 and fragment(s) thereof but that does not substantially recognize and bind other molecules in a sample, for example, a biological sample, which naturally includes a polypeptide of sFlt-1.

The detection reagents for determining the level of sFlt-1 or fragment(s) thereof, and optionally for determining the level of PAPP-A, PlGF and/or or fragment(s) thereof, are preferably selected from those necessary to perform the method, for example antibodies directed to sFlt-1, suitable labels, such as fluorescent labels, preferably two separate fluorescent labels suitable for application in the KRYPTOR assay, sample collection tubes

As used herein, the term “determining a level of sFlt-1 or fragment(s) thereof in a sample” refers to any means of determining sFlt-1 or a fragment thereof.

As used herein, “fragment” shall mean a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 813 or more nucleotides or 10, 20, 30, 40, 50, 60, 70, 80, 90, 100, 150, 186, 200, 250, 271 amino acids or more. Preferred fragments have sFlt-1 biological activity.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not having an infection and “disease” populations, e.g. subjects having an infection. For any particular marker (like sFlt-1), a distribution of marker levels for subjects with and without a disease/condition will likely overlap. Under such conditions, a test does not absolutely distinguish normal from disease with 100% accuracy, and the area of overlap might indicate where the test cannot distinguish normal from disease. A threshold is selected, below which the test is considered to be abnormal and above which the test is considered to be normal or below or above which the test indicates a specific condition, e.g. infection. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143:29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test. The AUC (area under the curve) makes it easy to compare one ROC curve to another. The ROC curve with greater the AUC represents a logistic regression.

As used herein, terms such as “marker”, “surrogate”, “prognostic marker”, “factor” or “biomarker” or “biological marker” are used interchangeably and relate to measurable and quantifiable biological markers (e.g., specific protein or enzyme concentration or a fragment thereof, specific hormone concentration or a fragment thereof, or presence of biological substances or a fragment thereof) which serve as indices for health- and physiology-related assessments, such as a disease/disorder/clinical condition risk, preferably an adverse event. A marker or biomarker is defined as a characteristic that can be objectively measured and evaluated as an indicator of normal biological processes, pathogenic processes, or pharmacologic responses to a therapeutic intervention. Biomarkers may be measured in a sample (as a blood, plasma, urine, or tissue test).

The stage of pregnancy at which the methods described herein may be practiced depends on various clinical factors including the overall health of the subject and the severity of the symptoms of preeclampsia.

In certain embodiments, the method is carried out on a subject no later than the end of 12th week of gestation. The end of 12^{th} week of gestation shall mean the last day of 12^{th} week of gestation, or the last second day, or the last third day or the last fourth day of the 12^{th} week of gestation.

In some embodiments, the method is carried out on a subject in the 9^{th} week of gestation. In one embodiment, the method is carried out in 10^{th} week of gestation. In one embodiment, the method is carried out in 11^{th} week of gestation. In embodiments, the method is carried out later than 12th week of gestation. In embodiments, the method is carried out in the 13^{th} week of gestation. In embodiment, the method is carried out in the 14^{th} week of gestation. In embodiment, the method is carried out between and including 15^{th} and 20^{th} week of gestation.

As used herein, “sample” shall mean a bodily fluid sample, such as a blood sample, such as a venous blood sample, a capillary blood sample, a serum sample, a plasma sample, a vaginal fluid sample, a saliva sample or an amniotic fluid sample, a cerebrospinal fluid preferably a blood, serum or plasma sample.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g.

“Serum” in the context of the present invention is the liquid fraction of whole blood that is collected after the blood is allowed to clot. When coagulated blood (clotted blood) is centrifuged serum can be obtained as supernatant.

The “sample” refers further to a tissue biopsy (e.g., placental tissue), chorionic villus sample, cell, or other specimen obtained from a subject. Desirably, the biological sample includes sFlt-1 nucleic acid molecules or polypeptides or both.

As used herein, the term “reference sample” is meant any sample, standard, or level that is used for comparison purposes. A “normal reference sample” can be a prior sample taken from the same subject, a sample from a pregnant subject not having any pregnancy related hypertensive disorder, such as preeclampsia or eclampsia, a subject that is pregnant but the sample was taken early in pregnancy (e.g., in the first or second trimester or before the detection of a pregnancy related hypertensive disorder, such as preeclampsia or eclampsia), a subject that is pregnant and has no history of a pregnancy related hypertensive disorder, such as preeclampsia or eclampsia, a subject that is not pregnant, a sample of a purified reference polypeptide at a known normal concentration (i.e., not indicative of a pregnancy related hypertensive disorder, such as preeclampsia or eclampsia).

As used herein, the term “reference level” refers to a value or number derived from a reference sample. A normal reference standard or level can be a value or number derived from a normal subject. Desirably, all reference samples, standard, and levels are matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of a pregnancy related hypertensive disorder, and a family history of a pregnancy related hypertensive disorder.

In one embodiment, the reference level is with respect to a value derived from a pregnant subject without developing a preeclampsia or pregnancy related hypertensive disorder (e.g., in the first or second trimester or before the detection of a pregnancy related hypertensive disorder, such as preeclampsia or eclampsia).

In embodiments, the reference level refers to a value derived form a pregnant subject with no history of a pregnancy related hypertensive disorder, such as preeclampsia or eclampsia.

In one embodiment, the reference levels and the level from a subject to be determined as used herein refers preferably to measurements of the protein level of sFlt-1 or fragments thereof in a blood sample, preferably a whole blood sample or plasma or serum sample obtained from a pregnant subject without developing a preeclampsia, by means of the Thermo Scientific B⋅R⊇A⋅H⋅M⋅S KRYPTOR Assay. Accordingly, the values disclosed herein may vary to some extent depending on the detection/measurement method employed, and the specific values disclosed herein are intended to also read on the corresponding values determined by other methods. In embodiments of the invention, the reduced level of sFlt-1 or fragment(s) thereof compared to the reference level that may define the transition from a low to a high risk of developing PE may be any drop in the range of 6% to 20% compared to the reference level. Any value within this range may be considered an appropriate reference level for high and low risk sFlt-1 levels. Furthermore, values below or equal to such a reference level may be indicative of high risk of preeclampsia, and values above such a reference level may be indicative of a low risk of preeclampsia. Appropriate cut-off levels that may be used in the context of the present invention, comprise, without limitation, at least 6%, 7%, 8%, 9%, 10%, 11%, 12%, 13%, 14%, 15%, 16%, 17%, 18%, 19%, 20%, 21%, 22, 23%, 24% or 25% change compared to the reference level.

As used herein, the term “positive reference” sample, standard or value is a sample or value or number derived from a subject that is known to have or to have had a pregnancy related hypertensive disorder, such as preeclampsia or eclampsia. The reference standard or level can also reflect the average or mean value of the level of the nucleic acid, polypeptide, or small molecule from normal reference subjects or positive reference subjects depending on the context. The reference can also be a chart, a graph, or a standard curve representing normal reference levels of the polypeptide, nucleic acid, or small molecule at any and/or all stages of pregnancy (e.g., weekly). Desirably, all reference samples, standard, and levels are matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of a pregnancy related hypertensive disorder, and a family history of a pregnancy related hypertensive disorder.

As used herein, the term “history of a pregnancy related hypertensive disorder” shall mean a previous diagnosis of a pregnancy related hypertensive disorder (e.g., preeclampsia or eclampsia or gestational hypertension) in the subject themselves or in a related family member.

As used herein, “gestational age” shall mean a reference to the age of the fetus, counting from the first day of the mother's last menstrual period. It refers also to the corresponding age of gestation as estimated by more accurate method in the art. In case of in-vitro fertilization 14 days adding to a known duration since fertilization. Gestational age can be determined by obstetric ultrasonography.

As used herein, “maternal age” shall mean the age of the pregnant subject at the time of delivery.

As used herein, the term “gestational hypertension” shall mean the development of high blood pressure without proteinuria after 20 weeks of pregnancy.

As used herein, the term “polypeptide” refers to a polymer of amino acids, and not to a specific length. Thus, peptides, oligopeptides and proteins are included within the definition of polypeptide.

The term “including” is used herein to mean, and is used interchangeably with, the phrase “including but not limited to”.

The term “such as” is used herein to mean, and is used interchangeably with, the phrase “such as but not limited to”.

As used herein, the term “risk parameter” or “risk factor” refers to the health conditions which predispose a pregnant subject to developing preeclampsia. One of the risk parameter is blood group of parent, preferably blood group of biological father or biological mother AB. A further preferred blood group is Rh factor of the parents, especially if the pregnant subject is Rh negative and the biological father of the fetus is Rh positive. The risk parameter includes but not limited to hypothyroidism, hyperthyroidism, BMI over 24, first pregnancy, history of preeclampsia, ethnic with impaired risk, multiple pregnancy, migraines, lupus, blood coagulation disorder such as increased clotting, inflammatory diseases, cardiac preliminary disorders, diabetes, chronic kidney disease, and chronic hypertension.

The method as described herein, the level of sFlt-1 below or equal to a reference level indicates initiating or modifying a treatment of the subject to decrease the risk of developing, delay the time point of the onset or at least reduce the severity of preeclampsia such as balancing the angiogenetic/anti-angiogenetic process in the placental development, lowering blood pressure, protect organ functions such as from the kidney or liver.

In embodiments, the level of sFlt-1 below or equal to a reference level indicates initiating or modifying a treatment of the subject to decrease the risk of developing, delay the time point of the onset or at least reduce the severity of preeclampsia such as balancing the angiogenetic/anti-angiogenetic process in the placental development, lowering blood pressure, protect organ functions such as from the kidney or liver. Such treatment is also related to antenatal surveillance, modification of lifestyle, nutritional supplementation, bed rest, restriction of activity or regular exercise, nutritional measures as reduced salt intake, and antioxidants such as vitamins C and E, garlic, marine oil.

As used herein, the term “nulliparous” refers to subject has never given birth. “Primiparous” shall mean the subject has given birth once. “biparous” shall mean the subject has given birth twice. “Multiparous” shall mean the subject has given birth more than twice.

As used herein, the term “chromosomal abnormality” shall mean difference in the chromosomes that can happen during development of the fetus. They could be unique to the fetus or inherited from a parent. Abnormalities are split into two categories: numerical referring to a different number of chromosomes than expected, such as monosomy or trisomy, and structural referring to translocation, deletion, duplication, formation of a ring as a result of a portion of a chromosome tearing off, inversion of chromosome. The chromosomal abnormalities include but not limited to Down Syndrome, Turner Syndrome, Klinefelter Syndrome, Trisomy 13, Trisomy 14, triple X Syndrome, XYY syndrome, Fragile X Syndrome, Cri-Du-Chat Syndrome.

The invention further relates to kits, the use of the kits and methods wherein such kits are used. The invention relates to kits for carrying out the herein above and below provided methods. The herein provided definitions, e.g. provided in relation to the methods, also apply to the kits of the invention. The kits can be part of a medical device which also contains calibrators, controls, buffer reagents and can be used in connection with a diagnostic instrument and/or software. In particular, the invention relates to kits for therapy monitoring, comprising the prognosis, risk assessment or risk stratification of a subsequent adverse event in the health of a patient, wherein said kit comprises the detection reagents for determining the level sFlt-1 or fragment(s) thereof, and optionally additional reagents for determining the level of further biomarker in a sample from a subject, and reference data, such as a reference level, corresponding to sFlt-1 risk levels, and optionally further biomarker levels, wherein said reference data is preferably stored on a computer readable medium and/or employed in in the form of computer executable code configured for comparing the determined levels of sFlt-1 or fragment(s) thereof, and optionally additionally the determined levels of further biomarker or fragment(s) thereof, to said reference data.

In one embodiment of the method described herein, the method additionally comprises comparing the determined level of sFlt-1 or fragment(s) thereof to a reference level, threshold value and/or a population average corresponding to sFlt-1 or fragments thereof in patients who is at risk of getting PE, wherein said comparing is carried out in a computer processor using computer executable code.

The methods of the present invention may in part be computer-implemented. For example, the step of comparing the detected level of a biomarker, e.g. the sFlt-1 or fragments thereof, with a reference level can be performed in a computer system. In the computer-system, the determined level of the biomarker(s) can be combined with other biomarker levels and/or clinical parameters of the subject in order to calculate a score, which is indicative for the prognosis, risk assessment and/or risk stratification. For example, the determined values may be entered (either manually by a health professional or automatically from the device(s) in which the respective marker level(s) has/have been determined) into the computer-system. The computer-system can be directly at the point-of-care (e.g. primary care, hospital or home setting) or it can be at a remote location connected via a computer network (e.g. via the internet, or specialized medical cloud-systems, optionally combinable with other IT-systems or platforms such as hospital information systems (HIS)). Typically, the computer-system will store the values (e.g. biomarker level or clinical parameters such as age, blood pressure, weight, sex, etc. or pregnancy parameter such as UAPI, FMF algorithms, scores such as VOCAL score, BMI etc.) on a computer-readable medium and calculate the score based-on pre-defined and/or pre-stored reference levels or reference values. The resulting score will be displayed and/or printed for the user (typically a health professional such as a physician or the patient). Alternatively, or in addition, the associated prognosis, assessment, treatment guidance, patient management guidance or stratification will be displayed and/or printed for the user (typically a health professional such as a physician or the patient).

In one embodiment of the invention, a software system can be employed, in which a machine learning algorithm is evident, preferably to identify patients at risk for PE using data from electronic health records (EHRs). A machine learning approach can be trained on a random forest classifier using EHR data (such as labs, biomarker expression, vitals, and demographics) from patients. Machine learning is a type of artificial intelligence that provides computers with the ability to learn complex patterns in data without being explicitly programmed, unlike simpler rule-based systems. Earlier studies have used electronic health record data to trigger alerts to detect clinical deterioration in general. In one embodiment of the invention the processing of sFlt-1 levels may be incorporated into appropriate software for comparison to existing data sets, for example sFlt-1 levels may also be processed in machine learning software to assist in prognosing the occurrence of PE.

“PAPP-A” is pregnancy-associated plasma protein A, pappalysin-1, and refers to a plasma protein that is used as a screening test between 8 and 14 weeks gestation. The diminished levels of the protein suggest an increased risk of Down Syndrome, intrauterine growth retardation, preeclampsia and stillbirth.

“PlGF” is placental growth factor (UniprotKB-Q6IB04), a member of the vascular endothelial growth factor (VEGF) family. PlGF is involved in the pathway glycosylphosphatidylinositol-achor biosynthesis, which is part of Glycolipid biosynthesis. The levels of PlGF drop in pregnant subject destined to develop preeclampsia.

As used herein, reference data, such as a reference level, corresponding to patient groups with maternal age up to 18, between 18-34, above 34, and optionally additional markers as described herein, preferably, PAPP-A and/or PlGF levels, wherein said reference data is preferably stored on a computer readable medium and/or employed in the form of computer executable code configured for comparing the determined levels of sFlt-1 or fragment(s) thereof, and optionally additionally the determined levels of PAPP-A and/or PlGF or fragment(s) thereof, to said reference data. “Reference date” includes further reference level corresponding to patient groups with blood group AB, with blood group Rh negative, with blood group Rh negative and biological father of the fetus Rh positive, carrying at least one female fetus, carrying at least one male fetus, nulliparous, having one or more former pregnancies, and/or being suspected of carrying a fetus with a chromosomal abnormality. The reference data can also include an instruction manual how to use the kits of the invention.

The kit may additionally comprise items useful for obtaining a sample, such as a blood sample, for example the kit may comprise a container, wherein said container comprises a device for attachment of said container to a cannula or syringe, is a syringe suitable for blood isolation, exhibits an internal pressure less than atmospheric pressure, such as is suitable for drawing a pre-determined volume of sample into said container, and/or comprises additionally detergents, chaotropic salts, ribonuclease inhibitors, chelating agents, such as guanidinium isothiocyanate, guanidinium hydrochloride, sodium dodecylsulfate, polyoxyethylene sorbitan monolaurate, RNAse inhibitor proteins, and mixtures thereof, and/or A filter system containing nitro-cellulose, silica matrix, ferromagnetic spheres, a cup retrieve spill over, trehalose, fructose, lactose, mannose, poly-ethylen-glycol, glycerol, EDTA, TRIS, limonene, xylene, benzoyl, phenol, mineral oil, anilin, pyrol, citrate, and mixtures thereof.

### FIGURES

The invention is further described by the figures. These are not intended to limit the scope of the invention.

FIG. 1: Boxplots representing levels of sFlt-1 before 90 days GA, between 90-100 days GA, or between 140-154 days GA, comparing levels in subjects with or without EO-PE. The study involved 11,952 women recruited in the first-trimester, 11,918 without PE, 34 with Early-onset PE. Before 90 days of gestation, all cases of women with EO-PE (N=10) had a sFlt-1 level below the median (p<0.01). Between 90 and 100 days of gestation, in average, women with EO-PE (N=24) had a sFlt-1 level in the average. Between 140 and 154 days of gestation, all women with EO-PE (N=4) had a sFlt-1 above the median.

FIG. 2: Statistical evaluation of sFlt-1 levels in subjects in which samples were obtained in the first trimester. The study involved 11,952 women recruited in the first-trimester, 11,918 without PE, 34 with Early-onset PE. As can be observed, SFlt-1 in the first trimester is not associated with EO-PE (EO-PE: 0.98 MoM vs controls: 1.00 MoM, p=0.09).

FIG. 3: Statistical evaluation of sFlt-1 levels in subjects in which samples were obtained after 12 6/7 weeks GA. The study involved 7,409 women recruited after 90 days of gestation, 7,385 without PE, 24 with Early-onset PE. As can be observed, sFlt-1 after 12 6/7 weeks is not associated with EO-PE (EO-PE: 1.00 MoM vs controls: 1.00 MOM, p=0.40).

FIG. 4: ROC curve of the data presented in FIG. 3, in which it is shown that sFlt-1 after 12 6/7 weeks is not associated with EO-PE.

FIG. 5: Statistical evaluation of sFlt-1 levels in subjects in which samples were obtained before 12 6/7 weeks GA (before 90 days GA). The study involved 4,543 women recruited before 90 days of gestation, 4,533 without PE, 10 with Early-onset PE. As can be observed, sFlt-1 before 12 6/7 weeks Is inversely associated with EO-PE (EO-PE: 0.94 MoM vs controls: 1.00 MoM, p=0.003).

FIG. 6: ROC curve of the data presented in FIG. 5, in which it is shown that sFlt-1 before 12 6/7 weeks correlates with EO-PE. Measurement of sFlt-1 (adjusted for gestational age-MoM) and sFlt-1 (not adjusted for gestational age-raw) are both predictive of early-onset PE and can be used as a marker of early-onset PE (AUC: 0.74 (95% CI: 0.64-0.84, p<0.01)).

FIG. 7: ROC curve of data for sFlt-1 and PlGF levels in subjects in which samples were obtained after 12 6/7 weeks GA (after 90 days GA). After 90 days of gestation PlGF is a strong marker of EO-PE, while sFlt-1 is not.

FIG. 8: ROC curve of data for sFlt-1 and PlGF levels in subjects in which samples were obtained before 12 6/7 weeks GA (before 90 days GA). Before 90 days of gestation: sFlt-1 is a strong marker of EO-PE, while PlGF is not as good as after 90 days.

FIG. 9: ROC curve of data for sFlt-1 and PlGF levels in subjects in which samples were obtained before 12 6/7 weeks GA (before 90 days GA). Additionally, a ROC curve is shown using combined data for both sFlt-1 and PlGF levels. The ROC curve shows AUC values for PlGF: 0.70 (95% CI: 0.56-0.85), sFlt-1:0.74 (95% CI: 0.64-0.84), and the combination of both markers: 0.85 (95% CI: 0.78-0.92).

FIG. 10: ROC curve of data for sFlt-1 and PlGF levels in subjects in which samples were obtained before 12 6/7 weeks GA (before 90 days GA). Additionally, data for uterine artery Doppler measurements as obtained in the study was combined in the analysis. A ROC curve is shown using combined data for both sFlt-1 and PlGF levels, and in addition, these combined levels are further combined with Doppler data. The ROC curve shows an AUC value for the Doppler combination of 0.87 (95% CI: 0.80-0.94).

FIG. 11: ROC curve of data for sFlt-1 and PlGF levels in subjects in which samples were obtained between 90- and 100-days GA. The combination of sFlt-1 and PlGF predicts some cases of IUFD between 90 and 100 days.

FIG. 12: ROC curve of data for sFlt-1 and PlGF levels in subjects in which samples were obtained before 90 days GA. The combination of sFlt-1 and PlGF predicts IUFD, whereby the association is stronger before 90 days of gestation (AUC: 0.72 95%, CI: 0.60-0.84), compared to between 90-100 days of gestation.

FIG. 13: Boxplot and ROC curve showing correlation between sFlt-1 levels (measured before 90 days GA) in subjects with estimated high or low risk determined using the FMF algorithm. As can be seen from the figure, patients with high risk as determined with the FMF algorithm have significantly lower sFlt-1 levels prior to 90 days GA.

### EXAMPLES

### Example 1

### Study Design:

Pregnant women were recruited at 11-14 weeks of gestation and followed until delivery. sFlt-1 was measured at recruitment using the Thermo Scientific B⋅R⊇A⋅H⋅M⋅S KRYPTOR and reported in multiple of median (MoM) adjusted for gestational age. Median levels of sFlt1 were compared between women who developed early-onset PE (<34 weeks); mid-onset PE (34-36 weeks); late-onset PE (37 weeks or greater); and no PE (controls). The area under the ROC curves (AUC) was used to estimate the potential predictive values of sFlt-1 for PE.

### Results:

We included 12,383 participants who delivered after 16 weeks of gestation, of which 33 (0.3%) developed early-onset PE; 65 (0.5%) and 400 (4.0%) developed late-onset PE. We observed that first-trimester sFlt-1 was lower in participants who developed early-onset or mid-onset PE (p=0.02), but more importantly, it was observed that the difference was mainly present in participants recruited earlier in pregnancy.

A significant difference of predictive value for early-onset PE between women who were recruited in the 11th week (AUC: 0.82; 95% CI: 0.72-0.92; p<0.001); in the 12th week (AUC: 0.62; 95% CI: 0.49-0.74; p=0.06) was observed; and in the 13^{th} week of pregnancy (AUC: 0.50; 95% CI: 0.32-0.67; p=0.97). A similar trend was observed for the prediction of mid-onset PE.

### Conclusion:

First-trimester maternal sFlt-1 is decreased in women who will develop PE before term (<37 weeks). Its predictive value is significantly improved when collected in or before the 12th week of gestation and this particularity could explain the contradictory results between previous studies.

### Example 2

### Study Design:

We conducted a secondary analysis of a prospective cohort study of nulliparous women recruited at 11 to 14 weeks of gestation. Maternal characteristics, mean arterial blood pressure, levels of maternal serum biomarkers (pregnancy-associated plasma protein-A, placental growth factor, 2 risk cutoffs (1 in 70 and 1 in 100 from the FMF algorithm, sFlt-1 and mean uterine artery pulsatility index were obtained to calculate the risk of preterm PE and term PE compared to the to the reference group, that developed no preterm or term PE. The detection rate, false-positive rate, and positive and negative predictive values were calculated and computed for term and preterm PE as placenta-mediated complication. Woman who had reported taking daily aspirin were excluded. PAPP-A, PlGF, sFlt-1 concentrations have been measured by using the Thermo Scientific B⋅R⊇A⋅H⋅M⋅S KRYPTOR automated assays. Preterm PE was defined as PE with delivery before 37 weeks of gestation, and early-onset PE referred to cases with delivery before 34 weeks of gestation. Analyses were conducted using SAS statistical software packages (version 9.3; SAS Institute Inc, Cary, NC). A type I error of 5% was considered in all analyses.

### Results:

We included 4575 participants with complete observations. 29 patients developed a preterm PE, while 194 woman developed a term PE and 3705 woman developed no placenta-mediated complication (reference group). The median sFlt-1 value of the reference group was 1023 μg/mL, the Q1-Q3 value was 771-1373 pg/mL. The median sFlt-1 value and Q1-Q3 for woman with developing term PE was 933 μg/mL (726-1221 pg/ml). Therefore the pregnant woman who developed a term PE showed a decrease of the median sFlt-1 level of 8.8% and a fold change (FC) of the medians of 0.91.

The median sFlt-1 value and Q1-Q3 for woman with developing preterm PE was 852 μg/mL (658-1095 pg/ml). Therefore the pregnant woman who developed a preterm PE showed a higher sFlt-1 decrease as the term PE group with a median sFlt-1 decrease at week 11-14 of gestation of 16.7% and a fold change (FC) of the median of 0.83 compared to the reference group.

The Q1 of the sFlt-1 level of the preterm PE compared to the reference group showed a FC of 0.85 with a decrease of 14.7%.

The Q1 of the sFlt-1 level of the term PE compared to the reference group showed a FC of 0.94 with a decrease of 5.8%.

The Q3 of the sFlt-1 level of the preterm PE compared to the reference group showed a FC of 0.8 with a decrease of 20%.

The Q3 of the sFlt-1 level of the term PE compared to the reference group showed a FC of 0.89 with a decrease of 11%.

### Conclusion:

Nulliparous women with a decrease of the sFlt-1 median value, detected between week 11-14 of gestation, of at least 8.8% have an increased risk of developing a term PE, whereby a decrease of the median value of sFlt-1 of at least 16.7% have an increased risk of developing a preterm PE.

### Example 3

### Prediction of Early Preeclampsia and Prematurity in the First Trimester of Pregnancy (PREDICTION Study):

Pre-eclampsia is a complication of pregnancy affecting 2 to 5% of pregnant women. It is one of the main causes of maternal and neonatal mortality and morbidity in the world. Early-onset preeclampsia is that which requires delivery before the 34^{th} week of pregnancy and is associated with very significant perinatal morbidity.

Online software (Fetal Medicine Foundation) combining biophysical (age, BMI, BP, history), ultrasound (uterine artery Doppler) and biochemical (PlGF & PAPP-A) factors measured in the first trimester of pregnancy is currently available and suggests that more than 90% of early preeclampsia can be predicted with a false-positive rate of less than 10%.

Additional patient groups such as patients under 18 years old, with some rare blood group such as AB, Rh negative, with multiple pregnancies, presence, having experienced one or more former pregnancies, presence of a malformative or polymalformative syndrome and/or suspected or presence of carrying a fetus with a chromosomal abnormality (very high nuchal translucency) are further included and investigated in the present discovery study for exploring the correlation between biomarker such as sFlt-1, PAPP-A or PlGF and preeclampsia.

### Objectives:

1) Validate the FMF screening tool between 11 and 13 weeks of pregnancy for early preeclampsia and other placenta-related pregnancy disorders (preterm preeclampsia, IUGR <3 percentile, perinatal death);
2) To compare the screening tool with and without the use of uterine artery Doppler measurement;
3) To explore the efficacy of potential markers (serum sFlt-1, serum ADAM-12, serum PP-13, placental and subplacental volume, placental vascularity) for the prediction of preeclampsia

### Methodology:

Prospective observational study. Nulliparous pregnant women with a single pregnancy and a fetus without lethal abnormality were recruited between 11 3/7 and 13 6/7 weeks of pregnancy. A lifestyle questionnaire was completed. BMI and BP measurement and a blood sample (20 ml) were taken. A Doppler ultrasound of the uterine arteries and a 3D evaluation of the placenta have completed the visit. A follow-up on medical records was performed approximately 1 month after the expected delivery date.

Serum was analyzed within 10 days of recruitment for PAPP-A, PlGF, sFlt1, fbHCG and AFP using the Thermo Scientific B⋅R⊇A⋅H⋅M⋅S KRYPTOR automated assays. Residual serum was stored at −80 Celsius for additional analyses (PP-13, ADAM-12, vitamin D) at the end of the project to evaluate the possibility of improving the prediction model with promising markers, including placental volume and vascularity assessed by 3D ultrasound. For each participant, a calculation of the risk of early preeclampsia and overall preeclampsia was calculated with the help of FMF software, but was disclosed to them. The optimal sensitivity and specificity of the tool will be evaluated using ROC curves.

We have estimated an incidence of 0.7% of early onset preeclampsia (<34 weeks) in our nulliparous population (Quebec & Montreal). We recruited 7554 women to demonstrate that the FMF screening tool is at least 80% sensitive and 90% specific when it is expected to be 95% sensitive and 92% specific.

### Objectives

### Main Objective

Validate the FMF early pre-eclampsia screening tool between 11 and 13 weeks of pregnancy.

### Secondary Objectives

1) To evaluate the performance of the FMF test in predicting all cases of pre-eclampsia and in predicting other placenta-related pregnancy disorders (early and severe pre-eclampsia, IUGR <3 percentile, perinatal death).
2) Compare the screening tool with and without uterine artery Doppler.
3) In the event that the FMF screening tool is not positively validated in our population; assess the predictive value of each biomarker individually and evaluate whether they could be used in a different prediction model.
4) Assess the predictive value of potential biomarkers of early pre-eclampsia (sFlt-1, PP13, ADAM-12, 25-OH-vitamin D, placental volume and placental/subplacental vascularity, sFlt-1).
5) To assess the predictive value of cervical measurement in the 1st^{ter}trimester of pregnancy for preterm delivery.

### I. Research Quote

This is a prospective observational study in nulliparous pregnant women who were recruited in the 1st^{ter} trimester of pregnancy at which time numerous biomarkers were collected and analyzed and followed until delivery to verify the presence of our primary and secondary outcomes.

### II. Population and Selection Criteria

Studies set of inclusion criteria:
Woman with live singleton pregnancy between 11 3/7 weeks and 13 6/7 weeks.
Nulliparous woman (with no previous pregnancies up to 20/7 weeks gestation, regardless of reason)

### Studies Set of Exclusion Criteria:

Female <18 years old
Woman unable to give informed consent (e.g., does not understand English or French).
Women who planned to give birth outside the participating centres (except for women giving birth at the Hôtel-Dieu de Lévis, who were eligible for the project since they will have consented via the consent form to have their file consulted in this institution)
Woman positive for HIV or hepatitis C or chronic hepatitis B (not cured)

### Studies Set of Further Inclusion Criteria:

Multiple pregnancy (women who had two fetuses and one of them has stopped growing is not eligible)
Presence of a fetal malformative or polymalformative syndrome
Presence of a nuchal translucency measurement ≥3.5 mm being associated with a very high risk of chromosomal abnormality and/or cardiac malformation and may influence the serum PAPP-A value.
Negative fetal heart on the day of the recruitment visit

### III. Conduct of the Study

### 11 3/7-13 6/7 Weeks

Visit with the Nurse (11 3/7-13 6/7 Weeks)

First, the research nurse collected the blood specimen (2×5 ml tubes, BD Vacutainer SST) by venipuncture. The tubes were gently inverted 5 times to allow the reagents to mix well with the blood. The tubes rested in an upright position in a dark box until the treatment (minimum 30 minutes, maximum 2 hours). In the second step, the research nurse measured the patient's blood pressure. The patient had to rest in a sitting position with uncrossed legs for 5 minutes without talking before the measurement. The blood pressure was taken simultaneously on both arms (sleeveless vest), three times by a pre-programmed Microlife electric blood pressure monitor (model 33603).

A questionnaire was completed with the patient in order to know her medical and obstetrical family history and her socio-economic background, including her date of birth, anthropometric measurements, tagabism, etc. (see attached questionnaire). The total meeting with the nurse was 30 minutes maximum.

Visit with the Technologist (11 3/7-13 6/7 Weeks)

Following the meeting with the nurse, all patients were interviewed by the research team's radiology technologist and certified for nuchal translucency measurement to perform the ultrasound acquisitions using a Voluson E8 Expert (GE Medical Systems, Milwaukee, WI, USA) equipped with a 4-8 MHZ transducer. The instrument settings were the same for all patients, i.e., “angio mode”=100; “smooth”=4/5; FRQ=low; quality=normal; density=6; “enhance”=16; balance=175; WMF=low 1; “actual power”=2 dB; “pulse repetition frequency”=0.6 kHz; gain color=−7.2 dB

An ultrasound was performed to confirm the eligibility criteria for the project:

1) to confirm gestational age using cephalocaudal length (CCL) and biparietal diameter if CCL is ≥77 mm.

2) The nuchal translucency measurement was performed according to the criteria of the Fetal Medicine Foundation. If the participant had a prescription for the nuchal translucency measurement, a report with the result was provided.

3) In the presence of a malformative syndrome, multiple pregnancy, nuchal translucency ≥3.5 mm and/or negative fetal heart, the patient was notified and may was seen by one of the physicians in charge of the project or his substitute. An ultrasound report with the result was sent to her referring physician.

The technologist completed the eligibility sheet accordingly. A participant who meets at least one of the exclusion criteria was treated as any other participant, i.e., her blood samples were analysed and all data already collected or to be collected until delivery was retained. All data from these participants were excluded from the main analyses.

The visit window between 11 3/7 and 13 6/7 weeks of pregnancy was important and was respected to ensure the validity of certain data (biochemical and ultrasound). Thus, if the ultrasound dating test confirms a gestational age:
a) <11^{3/7} weeks (LCC<45 mm): the visit was rescheduled to a date between 11 3/7 and 13 6/7 weeks. Blood sampling and ultrasound were repeated at this time. Both samples were kept but only the second sample was used for the main analysis. The first samples was analysed and compared with the second samples in the case-control study if a minimum number of these patients (n≥5) present with early preeclampsia.

The participant's willingness to return for a repeat visit or not was respected. If she refused the second visit, the data collected were retained but excluded from the main analysis.
b) >14 weeks (CCL>84 mm): Blood samples, ultrasound data and questionnaire data were retained as such. No further visits were scheduled. These patients were excluded from the analyses for the primary objective.

The following ultrasound measurements were then be performed for research purposes:
4) Using Doppler ultrasound, the left and right uterine arteries were visualized and a pulsatility index measurement was performed according to FMF criteria: The uterine arteries were examined at the level of the internal cervical bone and the pulsatility index was calculated automatically by the machine using the pulsatile flow curves of three subsequent and similar cardiac cycles. The measurement was made in sagittal and transverse and the difference between the two was evaluated on a sample of approximately 1000 cases to assess the reproducibility, duration and efficiency of the two techniques. The presence and absence of notch were noted on both sides (a notch is considered present if an early diastolic incisure occurs on each cycle).
5) 3D ultrasound of the placenta and subplacental region with and without Doppler. This examination tooks about 30 seconds.

At the end of recruitment, for case-control analysis, a technologist blinded to the clinical data performed the following volume measurements and calculations: Using VOCAL (Virtual Organ Computer-aided Analysis) and a sequence of 6 sections of the placenta, each rotated 30 degrees from the previous one, horizontally on planes A and B, the placental contour were manually drawn, taking care to exclude the uterine wall. Similarly, the volume of the subplacental myometrium was assessed, from the border between the placenta and the deciduo-myometrium to the full thickness of the myometrium (up to 1 cm thick). These volumetric acquisitions were measured the following variables:
a. Placental Volume
b. Placental Quotient (PQ=1/4 Placental Volume/LCC).
c. The vascularity index (VI), flow index (FI) and vascular flow index (VFI): of the placenta and the deciduo-myometrial region will be evaluated using the VOCAL software. The VFI represents the number of stained voxels in the volume studied (expressed as a percentage). The FI is the average color value of all stained voxels representing the average intensity of blood flow (expressed as an absolute value between 0 and 100). The VIF is the average value of the color of all voxels in the studied region (gray and colored, expressed as an absolute value between 0 and 100).
6) Cervical length: vaginally (endovaginal probe). This examination, performed at the CHU of Quebec City only, validated the prediction of preterm delivery also present in the FMF algorithm. It was not done systematically in all participants, but done on prescription by the attending physician.
7) Ultrasound acquisition of the abdominal area was also performed to measure a posteriori the thickness of visceral adipose tissue that lies between the medial border of the rectus abdominis muscle and the anterior wall of the abdominal aorta. This ultrasound measurement may have greater predictive power than BMI in predicting preeclampsia. This acquisition takes only a few seconds longer.

The total time required for all ultrasound acquisitions was 15-35 minutes. Research technologists were certified and licensed for nuchal translucency and cervical measurement by the Fetal Medicine

Foundation (FMF) and the PQDT21. They received a DVD or USB stick with the images of the fetus as a thank you for their participation.

### Monitoring During Pregnancy (34 0/7-35 6/7 Weeks)

A survey (in electronic form, emailed to the participant) was done at 34^{e} weeks of pregnancy. This validated if the patient's medication has changed during the pregnancy and confirmed if the pregnancy is proceeding normally, if there moved, or if there were any complications to date. An email reminder was sent automatically one week later if the survey was not completed. A telephone contact was made to those who have not responded to the survey afterwards.

Postpartum Follow-Up (6 Weeks after DPA)

A second electronic survey (emailed to the participant) was done at approximately 6 weeks after her expected delivery date. This allowed us to validate whether the participant or her baby have experienced any difficulties following the birth. Among other things, we wanted to know about cases of post-partum pre-eclampsia, rare cases of perinatal or maternal death (regardless of the hospital where these events occurred). An email reminder was sent automatically one week later, if the survey was not completed. A telephone contact was made to those who have not responded to the survey afterwards. A procedure was put in place to ensure that participants who have had a termination, in-utero death or other adverse complication (which was mentioned in the 34-week survey) are not contacted again at 46 weeks if it was not necessary, in order to avoid inconvenience.

Follow-Up at the End of the Pregnancy (1 Month after the DPA)

Data on the pregnancy (gestational hypertension, preeclampsia, perinatal death, etc.), delivery (gestational age, etc.) and newborn (sex, birth weight, etc.) were collected from the patient's medical record via the CristalNet software by the research nurses following delivery. In the rare event that patients gave birth in a non-participating centre (not CHUL, HSFA, or Lévis), patients were contacted to request their permission to obtain this information at the delivery site. A second review of the data was done, directly from the medical record, by a physician (EB, KG, FA or their substitute) for all cases where preeclampsia was suspected and cases who delivered before 37 weeks (<10%, i.e., less than 532 records retrieved from the CHU-Q archives and less than 228 records retrieved from the CHUSJ archives).

### Blood Specimens

Blood specimens were transported to the laboratory in a box at room temperature and were centrifuged less than 2 hours after puncture, but more than 30 minutes after puncture.

The serum collected after centrifugation (1200×g for 10 minutes at room temperature) was transferred into 1 ml aliquots and then used to measure PAPP-A, PlGF, sFlt-1, free bHCG and maternal serum AFP using commercial kits validated by the FMF. For the CHU de Québec (CHUL and HSFA), the samples were stored at 4° C. until the assay is performed within 24 hours. For the CHU Ste-Justine, the aliquots were be kept at −20° C. and sent to the CHU de Quebec twice a month by registered mail on dry ice, in order to be analyzed and preserved. Following the primary analysis (serum PAPP-A, PlGF and sFlt-1 assay), the remaining sera were stored frozen at −80° C. and used to measure PP13, ADAM12 and vitamin D in the case-cohort sub-study conducted at the end of the main study.

### Case-Cohort Study

A case-cohort study nested within the main cohort assessed the predictive effect of potential biomarkers on the prediction of early pre-eclampsia. Maternal serum sFlt-1, ADAM-12, PP-13 and vitamin D was measured using commercial kits in a randomly selected subgroup of women (approximately 236 women) and in all cases of early preeclampsia (approximately 45) at the end of the study. This same case-cohort was analyzed for the following variables: placental volume (PV), placental and subplacental vascularity index (IV), flow index (FI), and vascular flow index (VFI).

### IV. Judging Criteria

Primary endpoint: Pre-eclampsia requiring delivery before 34 weeks of pregnancy based on gestational age determined by dates of last menstrual period (DMD) or 11-13 week ultrasound if the latter demonstrated a difference ≥5 days with the DMD method. Preeclampsia diagnosed according to the following criteria: 1) gestational hypertension ≥140 systolic and/or ≥90 diastolic (on 2 occasions within 4 hours) combined with any of the following conditions: growth retardation <10^{e} percentile, thrombocytopenia <100, AST and/or ALT more than twice the normal value, diastolic blood pressure ≥110 mmHg, and/or proteinuria (≥2+ on a stick or more than 300 mg/24 hours).

### Secondary Endpoints:

Birth <37 weeks (spontaneous labour or PPROM before 37 weeks of pregnancy)
Birth <34 weeks (spontaneous labour or PPROM before 34 weeks of pregnancy)

### Preeclampsia:

Premature preeclampsia <37 weeks

Severe preeclampsia (with any of its conditions: 1) ≥160 mmHg systolic and ≥110 mmHg diastolic after 4 hours of rest; 2) proteinuria ≥5 g/24 h or ≥3+ on rod; 3) oliguria≤400 ml/24 hours; visual or cerebral disorders; epigastric pain; pulmonary edema or cyanosis; thrombocytopenia <100,000 mm3.

Perinatal death (antepartum, up to 7 days after birth)
Growth retardation (<10^{e} percentile), based on Canadian reference values
Severe stunting (<3^{e} percentile), based on Canadian reference values
Birth weight <2500 grams
Birth weight <1500 grams

### Average Birth Weight

V. Data analysis plan

Main analysis: At the end of the study, the calculation of the risk of early preeclampsia and preeclampsia were automatically performed by the FMF software using all necessary collected data, including uterine Doppler measurements. The risk calculation was repeated automatically for all eligible participants excluding the uterine Doppler measurement.

The area under the curve, sensitivity, specificity, positive predictive value and negative predictive value of the two screening methods were reported and calculated using an ROC curve and different cut-off values.

### Results:

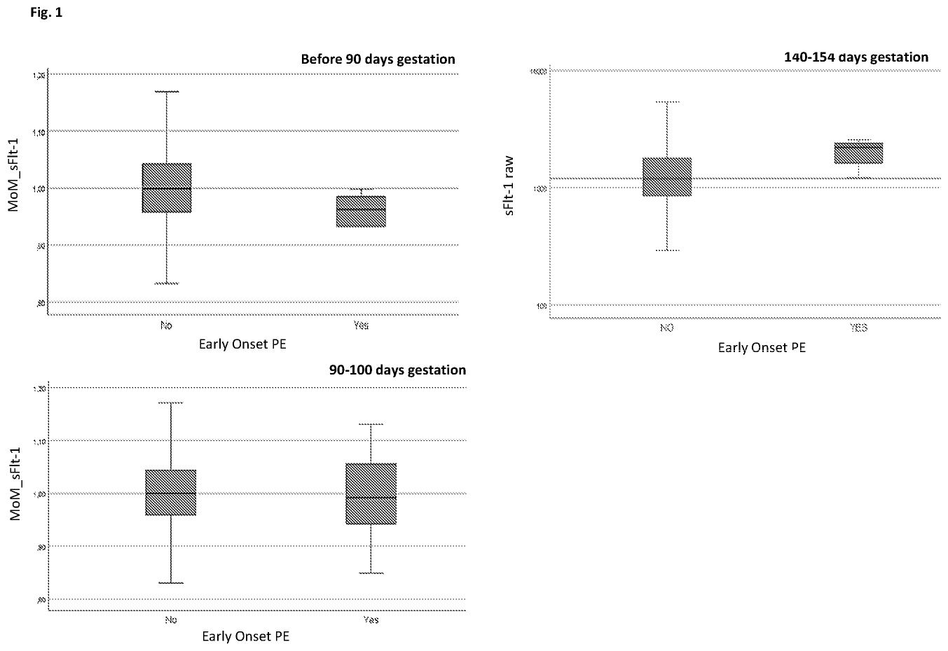

As can be seen from FIG. 1-12, the data obtained from the PREDICTION study support the prognostic capability of sFlt-1 in identifying patients at risk of EO-PE and/or IUFD when analysing samples obtained prior to 90 days GA.

As can be seen from FIG. 1, before 90 days of gestation, women with EO-PE (N=10) had a sFlt-1 level below the median (p<0.01). Between 90 and 100 days of gestation, in average, women with EO-PE (N=24) had a sFlt-1 level in the average. Between 140 and 154 days of gestation, women with EO-PE (N=4) had a sFlt-1 above the median. This study allows us to conclude that the level of sFlt-1 is abnormally decreased early in pregnancy (before 90 days of gestation) in women who will develop early preeclampsia (before 34 weeks) and will progressively increase to become normal at the end of the 1st trimester (between 90 and 100 days) and to become abnormally increased thereafter (after 140 days of gestation).

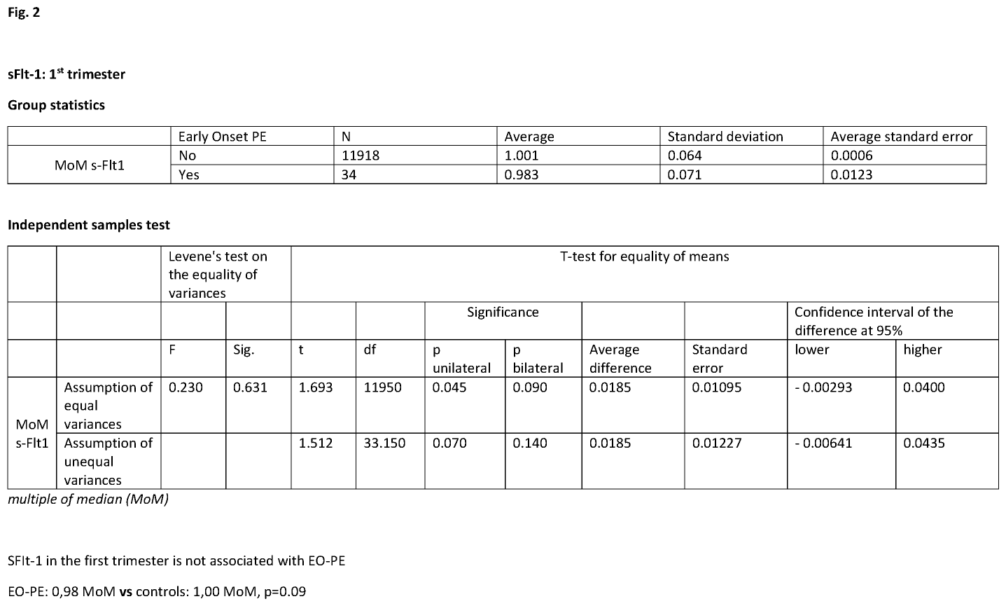

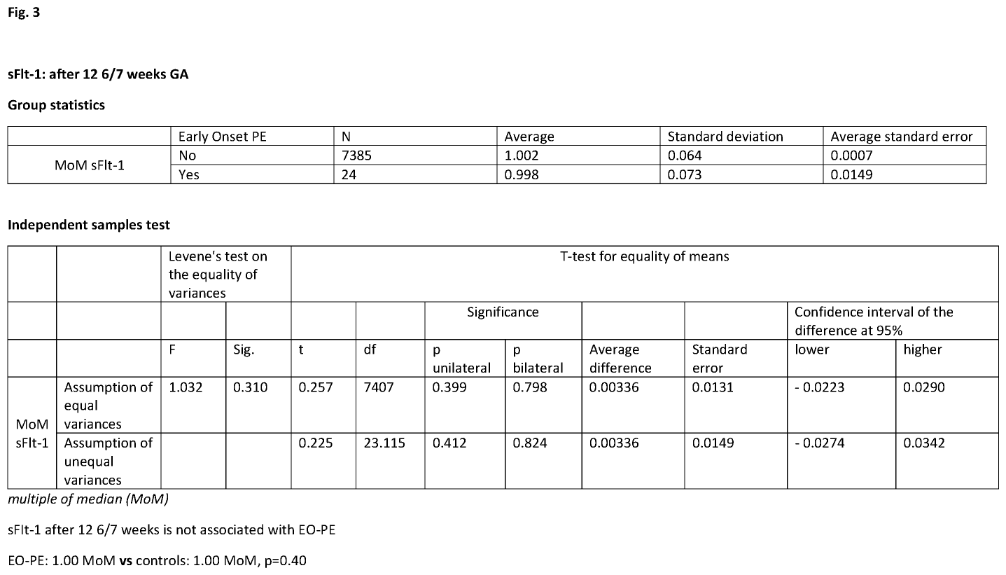

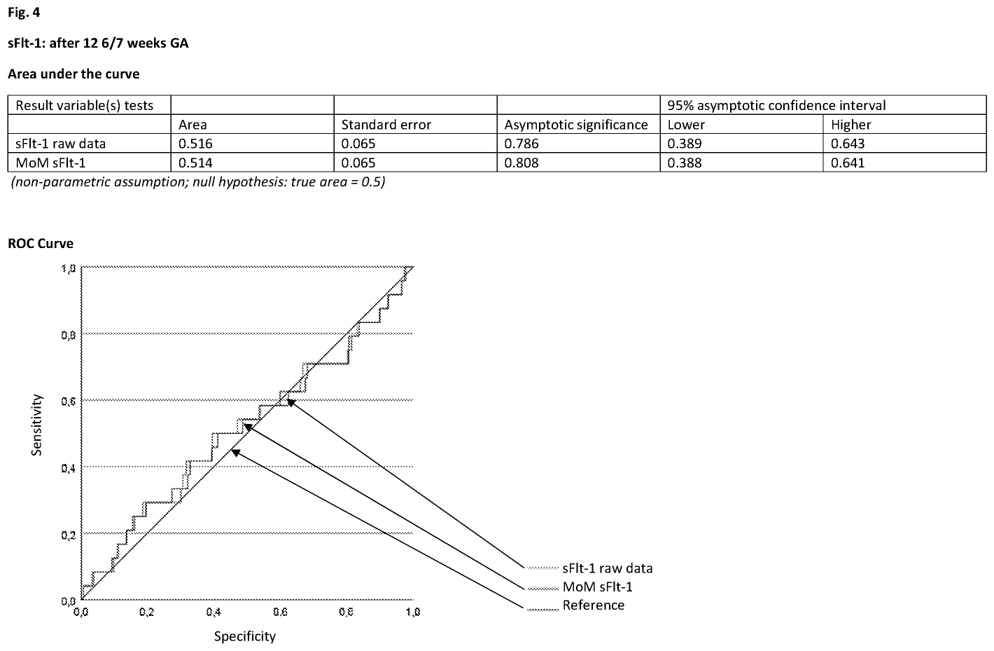

However, sFlt-1 measurement, when determined from samples obtained throughout the entire first trimester, does not provide a statistically relevant correlation with EO-PE (FIG. 2). Of further note is that sFlt-1 measurement, when determined from multiple samples obtained after 12 6/7 weeks of GA, also does not provide a statistically relevant correlation with EO-PE (FIG. 3, FIG. 4).

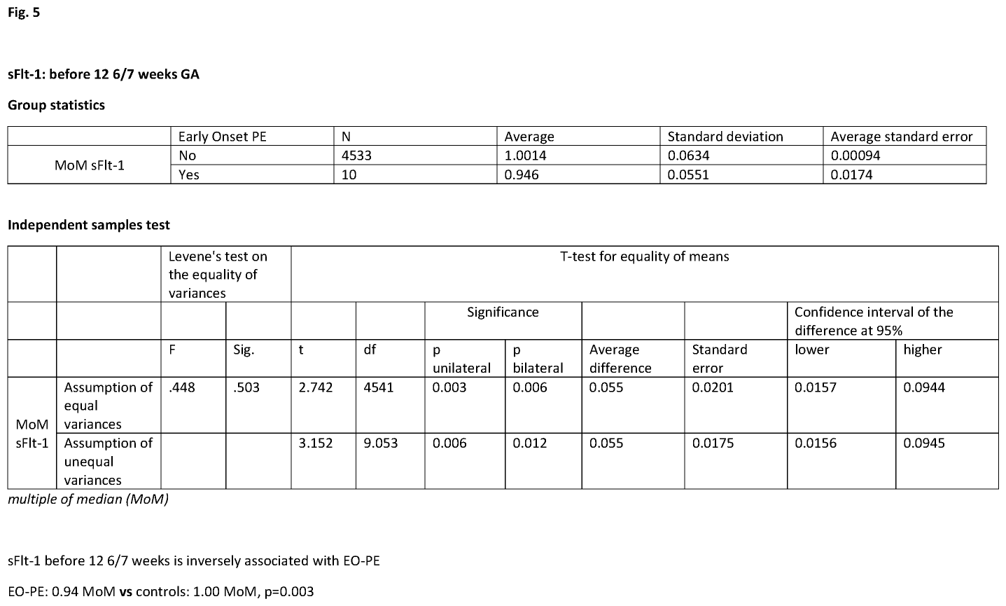

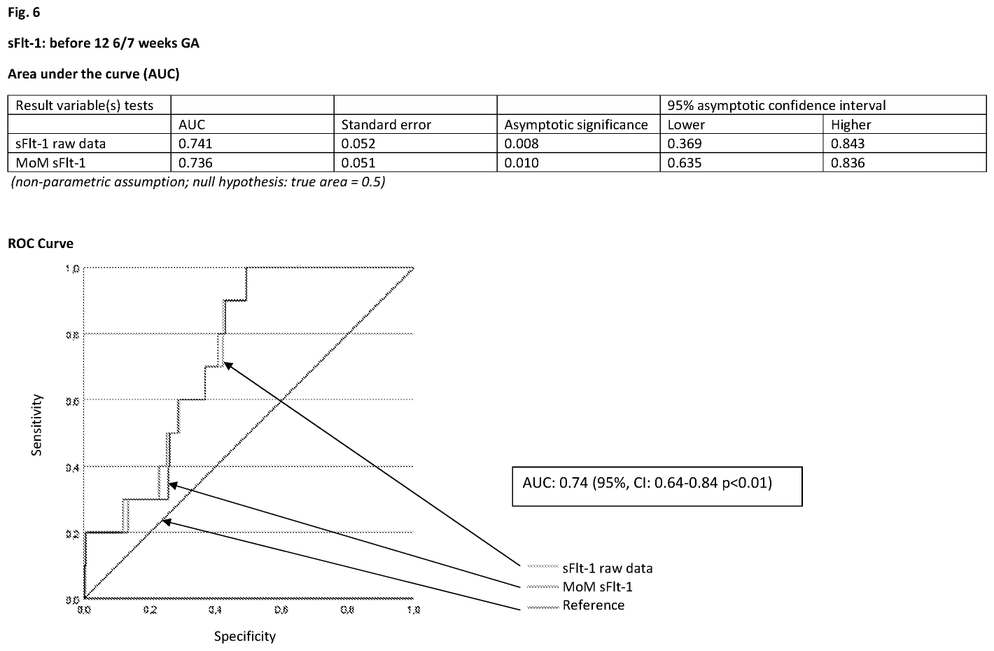

Surprisingly, the measurement of sFlt-1 within 12 weeks, or within 90 days of gestation, shows a significant correlation with EO-PE, in particular an inverse correlation with EO-PE (FIG. 5, FIG. 6). Thus, determining sFlt-1 within 90 days GA enables reliable prognosis of EA-PE at an early time point.

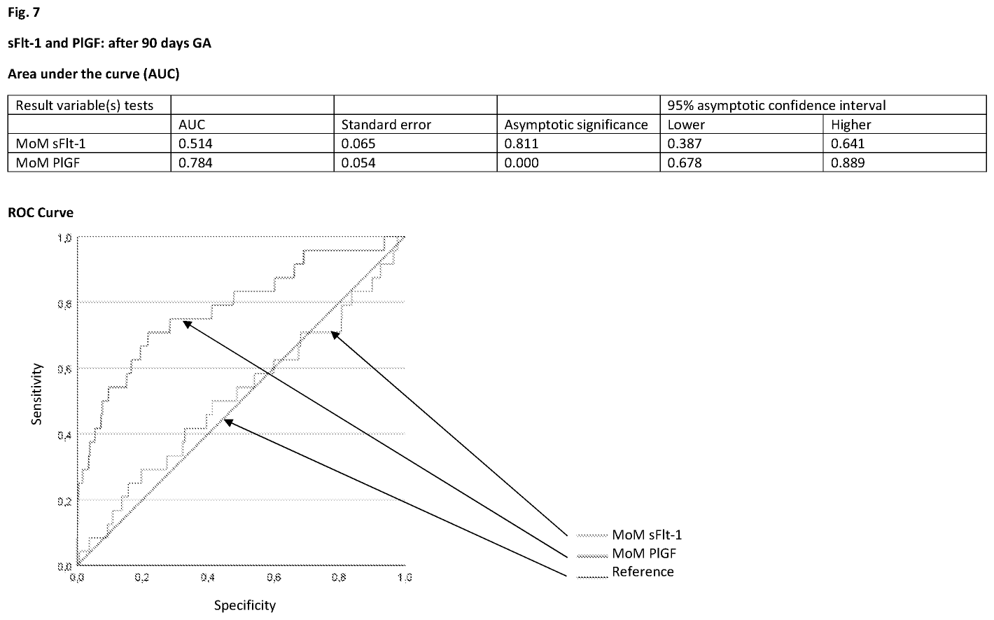

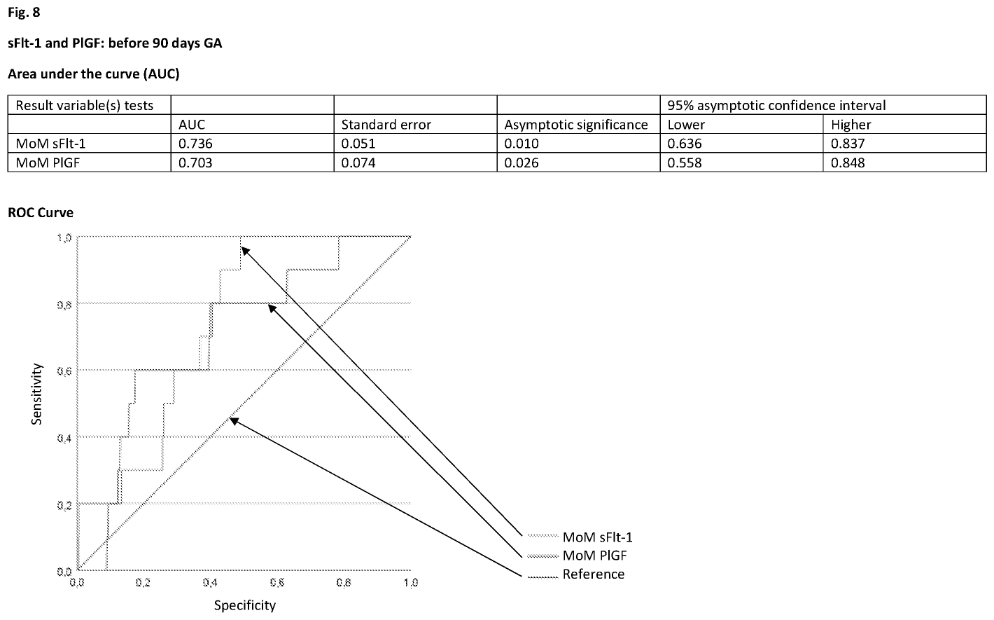

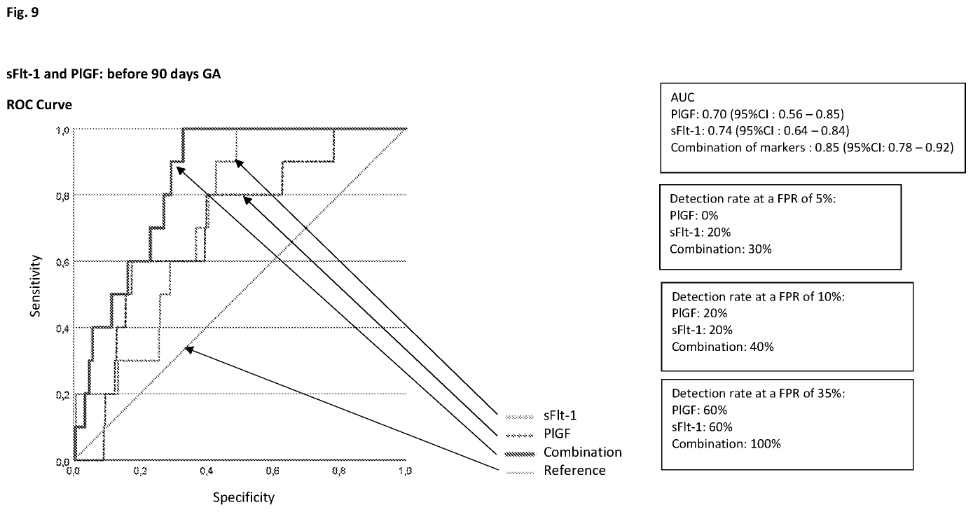

The combined use of sFlt-1 and PlGF leads to a statistically improved prognosis of EO-PE when samples are obtained early in pregnancy, for example before the end of the 12^{th} week of gestation (before 90 days GA). Of note, is that PlGF is typically effective in prognosing EO-PE when measured after 90 days GA, whereas sFlt-1 appears to enable no reliable prognostic statements from a single measurement after 90 days GA (FIG. 7). Surprisingly, both sFlt-1 and PlGF enable an EO-PE prognosis when measured before the end of 12 weeks (within 90 days) GA, although sFlt-1 appears to provide greater sensitivity at comparable specificity values, preferably above 0.6 (FIG. 8). Also surprisingly, the combined analysis of sFlt-1 and PlGF shows an unexpected and synergistic enhancement in EO-PE prognosis when measured before 90 days GA (FIG. 9).

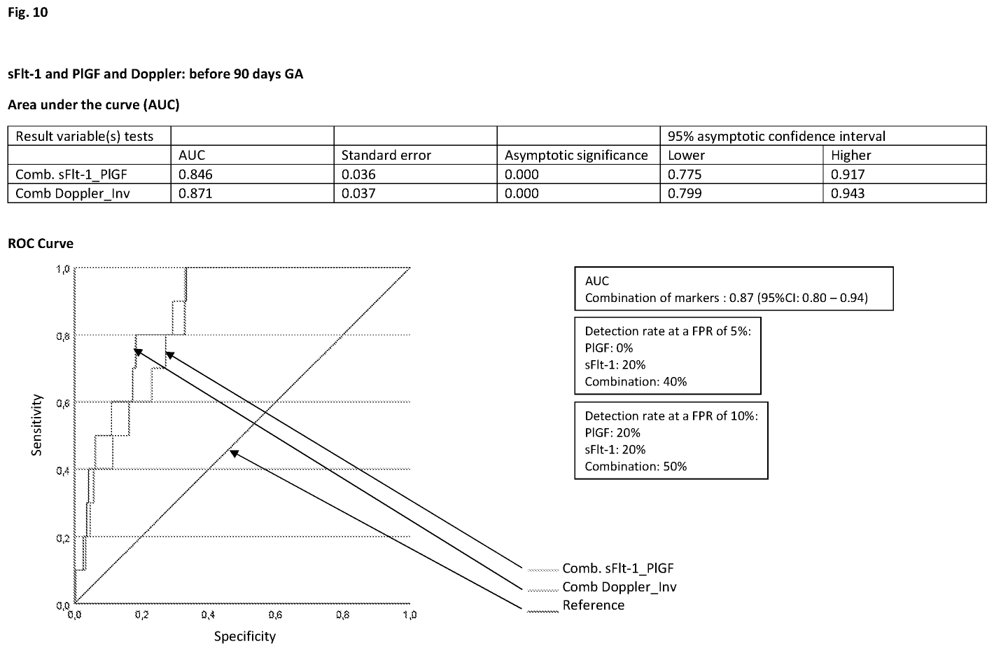

Also found through this study was that the combined use of uterine artery Doppler measurements, preferably in combination with sFlt-1, PlGF, maternal age and BMI, showed improved predictive power of EO-PE in subjects compared to sFlt-1 or PlGF alone (FIG. 10).

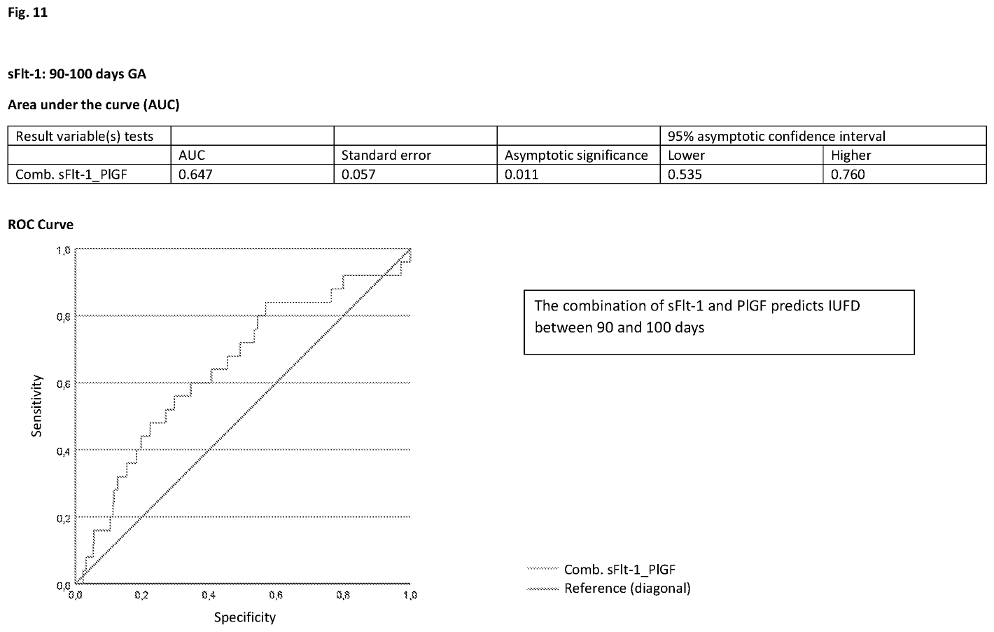

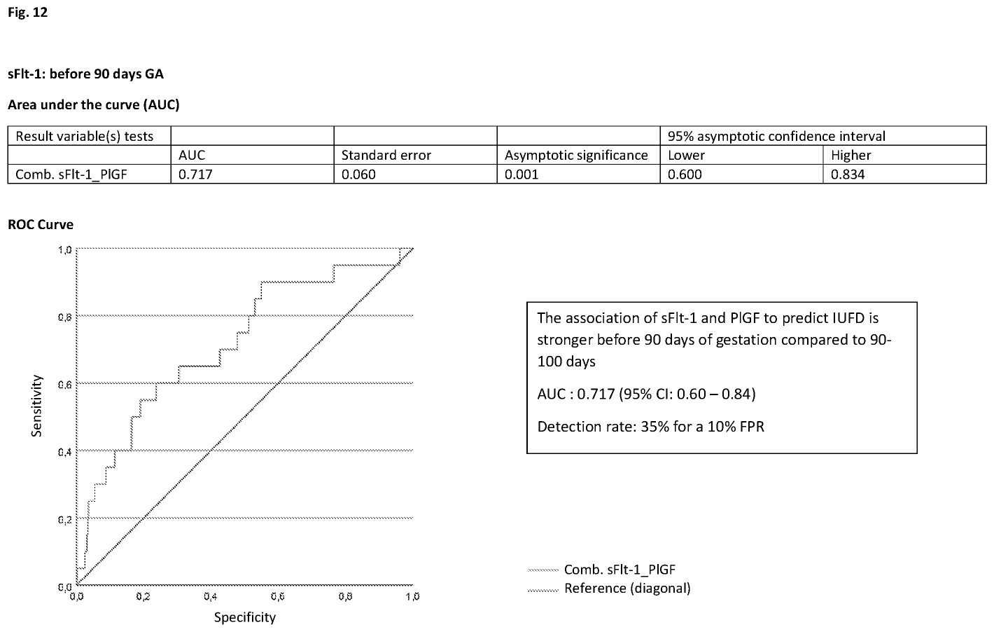

The combined use of sFlt-1 and PlGF also leads to a prognosis of IUFD when the sample is obtained between 90-11 days GA (FIG. 11), and shows statistically improved prognosis of IUFD when the sample is obtained before 90 days GA (FIG. 12).

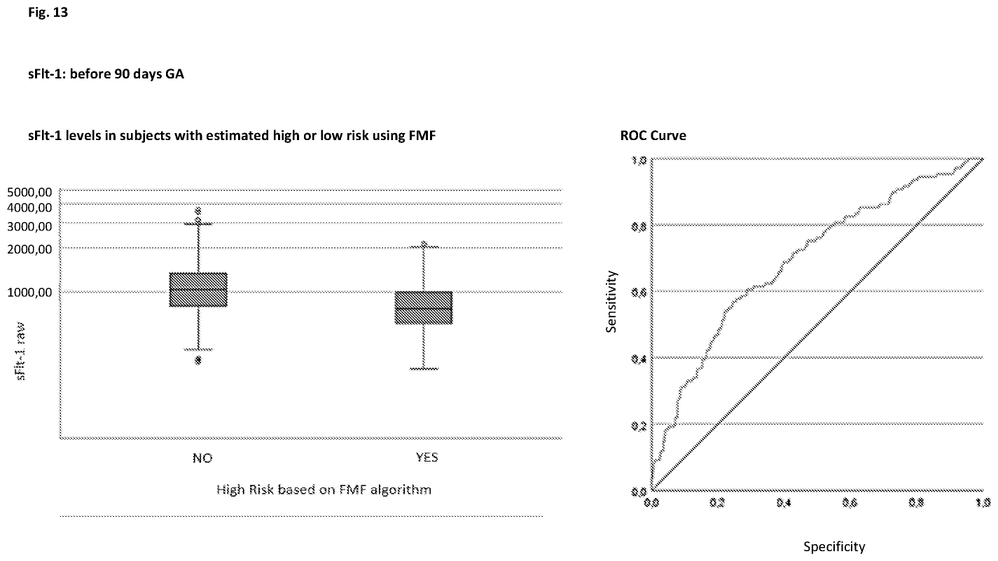

Additionally, comparisons in the data sets obtained using the FMF algorithm and the sFlt-1 analysis as described herein reveal that the two prognostic procedures reveal a strong correlation with each other. As can be seen from FIG. 13, patients with high risk as determined with the FMF algorithm have significantly lower sFlt-1 levels, prior to 90 days GA. Considering that the FMF screening algorithm includes consideration of multiple maternal characteristics and medical history, including factors such as blood pressure, pregnancy-associated plasma protein A and placenta growth factor, crown rump length, and uterine artery pulsatility index, the present findings represent a significant simplification in prognostic approaches that enable a comparable risk assessment thereby potentially avoiding the more complicated FMF approach. Analysis of sFlt-1 prior to 90 days may in fact enable prediction of pregnant women how would receive a positive FMF test, without the need for Doppler uterine artery assessment.

In conclusion, this study allowed us to conclude that sFLt-1 is decreased before 90 days of gestation in the majority of pregnant women who will develop early-onset preeclampsia whereas it will increase thereafter to become abnormally high in the 2nd trimester of pregnancy in the same pregnant women. Thus, sFlt-1 before 90 days of pregnancy allows to predict early-onset PE, and more particularly when combined with PlGF and/or uterine artery Doppler (Detection rate of 40% for a FPR of 10%). Furthermore, we also observed that the combination of the two markers could also predict about 35% of UFDIs for a false-positive rate of 10%.

The importance of this information is significant because the earlier aspirin or similar therapies in the first trimester are started in attempting to address and potentially avoid EO-PE, the more effective the treatment. The present invention thus enables an alternative and improved diagnostic approach to identifying patients at risk of EO-PE by using biomarker analyses of samples obtained early in pregnancy, as early as prior to 90 days GA, and subsequent therapy initiation and guidance.

1. A method for treating a subject to reduce the risk of early onset preeclampsia in a pregnant subject, comprising:
a. determining a level of soluble fms-like tyrosine kinase-1 (sFlt-1) or fragment(s) thereof in a sample that has been isolated from said pregnant subject,
b. wherein the sample is isolated from a subject before the end of the 12th week of gestation,
c. wherein said level of sFlt-1 or fragment(s) thereof is indicative of early onset preeclampsia occurring before the end of the 33rd week of gestation, and
d. treating said subject to decrease the risk of developing, delay the time point of the onset and/or reduce the severity of preeclampsia.

2. The method according to claim 1, comprising additionally:
a. determining a level of placental growth factor (PlGF) or fragment(s) thereof in the sample that has been isolated from the subject,
b, wherein a combination of the level of sFlt-1 or fragment(s) thereof and said level of PlGF or fragment(s) thereof is indicative of early onset preeclampsia occurring before the end of the 33rd week of gestation.

3. The method according to claim 1, wherein the subject is in the 9th to 11th week of gestation.

4. The method according to claim 3, wherein the subject is in the 11th week of gestation.

5. The method according to claim 1, comprising additionally:
a. determining or providing maternal age, body mass index and/or a uterine artery doppler measurement of the subject,
b, wherein the combination of levels of sFlt-1 or fragment(s) thereof, with maternal age, body mass index and/or a uterine artery doppler measurement of the subject, indicates early onset preeclampsia occurring before the end of the 33rd week of gestation.

6. The method according to claim 1, comprising additionally:
a. determining or providing a level of mean arterial pressure (MAP) of the subject,
b. wherein the combination of levels of sFlt-1 or fragment(s) thereof, with a level of MAP of the subject, indicates early onset preeclampsia occurring before the end of the 33^{rd} week of gestation.

7. The method according to claim 1, comprising:
a. determining a level of sFlt-1 or fragment(s) thereof, and determining a level of PlGF or fragment(s) thereof, in a sample that has been isolated from the subject, and
b. determining or providing maternal age, body mass index (BMI) and a uterine artery doppler measurement, and optionally mean arterial pressure (MAP), of the subject,
c. wherein a combination of said level of sFlt-1 or fragment(s) thereof, said level of PlGF or fragment(s) thereof, and maternal age, body mass index (BMI) and a uterine artery doppler measurement, and optionally mean arterial pressure (MAP), of the subject, is indicative of the likelihood of early onset preeclampsia occurring before the end of the 33^{rd} week of gestation.

8. The method according to claim 1, comprising:
a. determining a level of sFlt-1 or fragment(s) thereof, and determining a level of PlGF or fragment(s) thereof, in a sample that has been isolated from the subject, and
b. determining or providing maternal age, body mass index (BMI) and mean arterial pressure (MAP), and optionally a uterine artery doppler measurement of the subject,
c. wherein a combination of said level of sFlt-1 or fragment(s) thereof, said level of PlGF or fragment(s) thereof, and maternal age, body mass index (BMI) and mean arterial pressure (MAP), and optionally a uterine artery doppler measurement, of the subject, is indicative of the likelihood of early onset preeclampsia occurring before the end of the 33^{rd} week of gestation.

9. The method according to claim 1, wherein the level of sFlt-1 or fragment(s) thereof determined in the sample is compared to a reference level, selected from a population average and/or median for a healthy population, wherein a level of sFlt-1 or fragment(s) thereof below or equal to the reference level is indicative of high risk of early onset preeclampsia and treating said subject.

10. The method according to claim 2, wherein the level of PlGF or fragment(s) thereof determined in the sample is compared to a reference level, selected from a population average and/or median for a healthy population, wherein a level of PlGF or fragment(s) thereof above the reference level is indicative of high risk of early onset preeclampsia and treating said subject.

11. The method according to claim 1, wherein the level of sFlt-1 or fragment(s) thereof, indicates an early onset of preeclampsia occurring from begin of 20^{th} week of gestation and end of 33rd week of gestation.

12. The method according to claim 1, wherein the level of sFlt-1 or fragment(s) thereof, indicates additionally the subsequent occurrence of intra-uterine fetal death (IUFD).

13. The method according to claim 1, wherein the maternal age of the subject is 18-34, or above 34.

14. The method according to claim 1, wherein the sample is a bodily fluid sample selected from the group consisting of a venous blood sample, a capillary blood sample, a serum sample, a plasma sample, a vaginal fluid sample, a saliva sample and an amniotic fluid sample.

15. The method according to claim 1, wherein the level of sFlt-1 or fragment(s) thereof, and optionally a level of PlGF or fragment(s) thereof, maternal age, body mass index (BMI) a uterine artery doppler measurement, and/or mean arterial pressure (MAP) of the subject, indicates initiating or modifying a treatment of the subject to decrease the risk of developing, delay the time point of the onset and/or reduce the severity of preeclampsia, by balancing an angiogenetic/anti-angiogenetic process in placental development, lowering blood pressure and/or protect organ functions.

16. The method according to claim 15, wherein the treatment is selected from the group consisting of one or more diuretics, beta-blockers, ace inhibitors, angiotensin II receptor blockers, calcium channel blockers, alpha-blockers, methyldopa, central agonists, and vasodilators, VEGF, PLGF, statins, arginine vasopressin receptor antagonist, L-arginine, citrulline, inhibitor of arginase (nor-NOHA), iron chelating agent, heparin, magnesium sulphate, diazepam, phenytoin, vitamin D, calcium, selenium inhibition of molecules, extracorporal extraction, life style recommendations, ambulant monitoring, increase the frequency of maternal and fetal monitoring.

17. The method according to claim 16, wherein the treatment comprises administration of acetylsalicylic acid.

18. The method according to claim 1, wherein the subject is nulliparous.

19. The method according to claim 1, wherein the subject had one or more former pregnancies.

20. The method according to claim 1, wherein the subject has a multiple pregnancy.

21. The method according to claim 1, wherein the subject is suspected of carrying a fetus with a chromosomal abnormality.

22. The method according to claim 1, wherein the subject has one or more risk factors, selected from the group consisting of hypothyroidism, hyperthyroidism, BMI over 24, first pregnancy, history of preeclampsia, ethnic with impaired risk, multiple pregnancy, migraines, lupus, blood coagulation disorder, inflammatory diseases, cardiac preliminary disorders, diabetes, chronic kidney disease, and chronic hypertension.

23. The method according to claim 1, additionally comprising determining a level of at least one additional biomarker or fragment(s) thereof in a sample from said patient, wherein the at least one additional biomarker is selected from the group consisting of beta hCG, Copeptin, Vasopressin, Troponin, BNP, ANP, CRP, trombocytes/leucocytes, IL6, IL11, MR-proADM, VEGF, PAPP-A, PlGF, Endoglin, pro-Epil, PP-13, ADAM-12, Vitamin D, Inhibin-a, Activin-a, Pentraxin-3, p-Selectin, free fetal Hemoglobin, alpha-1-Microglobulin, unconjugated Estriol, alpha-Fetoprotein, GDF15, Neurophysin2, LNPEP, ESM1, HGF, pikachurin, hemopexin, pp13, uE3, CT-proET1, ADAM12, sTNFαR1, RBP4, ICAM, cell free fetal DNA, FSTL3, Visfatin, AFP, MMP9, TIMP1, Flt1, PCT, SHGB, Creatinine, GBP1, IGFALS, urine protein, PAI1/PAI2, catechol-o-methyltransferase (COMT), breakdown products of heme (bilirubin, biliverdin, carbon monoxide, ferritin), breakdown products of arginine, Uterine artery Doppler (UtA-Pi), diastolic Notch, MAP, blood pressure, smoking, leptin, genetic information and Arginine, wherein the level of the at least one additional biomarker and the level of sFlt-1 or fragment(s) thereof is indicative of early onset preeclampsia occurring before the end of the 33rd week of gestation.

24. A kit for carrying out the method of claim 1, comprising
detection reagents for determining a level of sFlt-1 or fragment(s) thereof and for determining a level of PlGF or fragment(s) thereof in a sample from a subject, and
a computer readable medium and/or computer software in the form of computer executable code, configured to:
i. compare a determined level of sFlt-1 or fragment(s) thereof and a determined level of PlGF or fragment(s) thereof, to one or more reference levels corresponding to a population average and/or median for a healthy population, and
ii. compare maternal age, body mass index, MAP and/or a uterine artery doppler measurement of the subject, to one or more reference levels, corresponding to a population average and/or median for a healthy population.

25. A method for determining a level of soluble fms-like tyrosine kinase-1 (sFlt-1) or fragment(s) thereof in a sample that has been isolated from a pregnant subject suspected to be at risk of early onset preeclampsia, comprising:
a. determining a level of soluble fms-like tyrosine kinase-1 (sFlt-1) or fragment(s) thereof in a sample that has been isolated from said pregnant subject,
b. wherein the sample is isolated from a subject before the end of the 12th week of gestation.

26. A method for treating a subject to reduce the risk of early onset preeclampsia in a pregnant subject, comprising:
a. determining a level of soluble fms-like tyrosine kinase-1 (sFlt-1) or fragment(s) thereof in a sample that has been isolated from said pregnant subject,
b. wherein the sample is isolated from a subject before the end of the 12th week of gestation, and
c. treating said subject to decrease the risk of developing, delay the time point of the onset and/or reduce the severity of preeclampsia.

27. A complex comprising a binder against soluble fms-like tyrosine kinase-1 (sFlt-1) or fragment(s) thereof, bound to sFlt-1 or fragment(s) thereof, in a sample that has been isolated from a pregnant subject suspected to be at risk of early onset preeclampsia, wherein the sample is isolated from a subject before the of the 12th week of gestation.
